(19) United States
(12) Patent Application Publication
(10) Pub. No.: US 2023/0329631 A1
(43) Pub. Date: Oct. 19, 2023

(54) SYSTEMS AND METHODS INVOLVING SLEEP MANAGEMENT

(71) Applicant: SRI International, Menlo Park, CA (US)

(72) Inventors: Massimiliano de Zambotti, Sunnyvale, CA (US); Fiona Baker, San Jose, CA (US); Devin Prouty, Menlo Park, CA (US); Ian Colrain, Redwood City, CA (US); Bhaskar Ramamurthy, Los Altos, CA (US); Glen W. McLaughlin, San Carlos, CA (US)

(73) Assignee: SRI International, Menlo Park, CA (US)

(21) Appl. No.: 17/906,699

(22) PCT Filed: Jun. 29, 2021
(86) PCT No.: PCT/US2021/039631

§ 371 Date: Sep. 19, 2022

Related U.S. Application Data
(60) Provisional application No. 63/045,304, filed on Jun. 29, 2020.

(51) Int. Cl. A61B 5/00; A61B 5/024; A61B 5/256

(52) U.S. Cl. CPC A61B 5/4809; A61B 5/4836; A61B 5/02405; A61B 5/256; A61B 5/02427; A61B 2562/0219

(57) ABSTRACT

Example embodiments are directed systems including processing circuitry and memory circuitry to store a predictive data model indicative of different patterns and probabilities of a user transitioning from an awake state to a sleep state. The processing circuitry is to detect, using data indicative of a current psychophysiological state of the user, a pattern among the different patterns of the predictive data model that is indicative of a probability of the user transitioning from the awake state to the sleep state at a date and time, based on the detected pattern, select an intervention action predicted to increase the probability of the user transitioning to the sleep state at the date and time, and communicate a message indicative of the intervention action to the user.

### BACKGROUND

Sleep is a biological need for health and wellbeing of an individual. In modern society, insufficient sleep is an epidemic. About one-third of the United States population has trouble sleeping, a proportion that is rising, with psychosocial stress being a recognized factor associated with poor sleep. Furthermore, in modern society, people report high levels of stress, worry, and concerns resulting in high pre-sleep psychophysiological activation (“hyperarousal”) which prevents an individual from falling asleep and obtaining a restful night.

### SUMMARY OF THE INVENTION

The present invention is directed to overcoming the above-mentioned challenges and others related to managing sleep for a user.

Various embodiments of the present disclosure are directed to systems, devices and methods thereof that can be used for managing sleep for a user by monitoring and/or manipulating factors which impact sleep and transition time, and providing intervention actions to improve sleep, such as to improve falling asleep (e.g., smoother physiological changes in the wake-to-sleep transition), to improve the sleep processes (e.g., reducing the arousal threshold, increase sleep efficiency, enhance nighttime cardiovascular function) and/or to reduce the transition time (e.g., faster perceived and/or physiological sleep onset latencies), sometimes referred to as a “wake-to-sleep transition time”. In some embodiments, the systems, devices, and/or methods thereof can additionally or alternatively be used for managing acute state of exaggerated psychophysiological activation and/or manipulating acute psychophysiological state.

Specific embodiments are directed to a system including processing circuitry and memory circuitry. The memory circuitry is to store a predictive data model indicative of different patterns and probabilities of a user transitioning from an awake state to a sleep state. The processing circuitry is to detect, using data indicative of a current psychophysiological state of the user, a pattern among the different patterns of the predictive data model that is indicative of a probability of the user transitioning from the awake state to the sleep state at a date and time. Further, the processing circuitry is to, based on the detected pattern, select an intervention action predicted to increase the probability of the user transitioning to the sleep state at the date and time, and communicate a message indicative of the intervention action to the user. The increased probability of the user transitioning to the sleep state at the date and time can optionally include an increased probability of decreasing the transition time to the sleep state for the user as compared to when no intervention action is taken.

In some embodiments, the processing circuitry is to detect the pattern by identifying, in the data, a feature set from among a plurality of feature sets and selecting a sub-model of the predictive data model using the feature set. The predictive data model can include a plurality of sub-models that indicate the probability of the user transitioning to the sleep state in response to different intervention actions, and the plurality of sub-models being associated with a particular feature set of the plurality of feature sets. Each feature of the particular feature set and/or of each of the plurality of feature sets can have a weight associated with the probability of the user transitioning to the sleep state. In some embodiments, the plurality of sub-models is associated with different time frames.

In some embodiments, the processing circuitry is to revise the predictive data model based on feedback data which is indicative of whether the user transitions to the sleep state responsive to the intervention action.

In some embodiments, the processing circuitry is to receive the feedback data in real time, and in response to the feedback data and the revised predictive data model, communicate another message indicative of a revised intervention action. For example, the processing circuitry can receive the feedback data, and in response to the received feedback data: identify features from the feedback data; identify whether the user exhibits a response to the intervention action that is anticipated by the predictive data model to increase the probability based on the identified features; and in response to an unexpected response, revise the predictive data model for the user and as associated with the detected pattern. The revised intervention action can be for a currently occurring sleep session (e.g., adjust in real-time) and/or for a future sleep session.

In some embodiments, the intervention action is part of a sleep intervention strategy that includes a plurality of intervention actions. The plurality of intervention actions can be selected from a group consisting of: a behavioral intervention action, a cognitive intervention action, a neuromodulation action, an environmental change, a sensory action, and combinations thereof.

In some embodiments, the processing circuitry is to communicate the message that is indicative of the sleep intervention strategy and which includes an order of the plurality of intervention actions.

In some embodiments, the processing circuitry is to communicate a plurality of messages, including the message, that are indicative of the plurality of intervention actions. Each of the plurality of messages can be selected from a group consisting of: a message displayed to the user that instructs the user to take a respective intervention action, and a message to another device to automatically cause a respective intervention action to occur at a particular time in accordance with the sleep intervention strategy.

In some embodiments, the system further includes input circuitry to receive the data indicative of the current psychophysiological state of the user. The input circuitry can include sensor circuitry, such as a wearable physiological sensor to sense a physiological signal from the user and another sensor to sense an atmospheric measurement.

In some embodiments, the input circuitry is to receive the data indicative of the current psychophysiological state of the user, wherein the data received is selected from a group consisting of: schedule or calendar data, stress level, general mood, dietary data, health information, exercise data, sleep data, and a combination thereof.

Various example embodiments are directed to a non-transitory computer-readable storage medium comprising instructions that when executed cause processing circuitry to: identify a feature set among a plurality of feature sets from data, the data being indicative of a current psychophysiological state of a user; based on the identified feature set, detect a pattern associated with a predictive data model that is indicative of a probability of the user transitioning from an awake state to a sleep state at a date and time; based on the detected pattern and the predictive data model, communicate a message to the user indicative of an intervention action predicted to increase the probability of the user transitioning to the sleep state at the date and time; and revise the predictive data model based on feedback data which is indicative of whether the user transitions to the sleep state in response to the intervention action.

In some embodiments, the instructions to detect the pattern include instructions executable to select a sub-model of the predictive data model associated using the identified feature set. The instructions can further be executed to, in response to the feedback data and the revised predictive data model, communicate another message indicative of a revised intervention action.

In some embodiments, the instructions to revise the predictive data model include instructions executable to revise a weight associated with the intervention action in response to the identified feature set for the user. The weight for the intervention action can be associated with a probability of the intervention action improving sleep and/or reducing a transition time to the sleep state. For example, the intervention action may improve the probability of falling asleep and/or the sleep process.

In some embodiments, the instructions to revise the predictive data model include instructions executable to revise the predictive data model over time for the user based on the feedback data and additionally received feedback data that is indicative of different sleep intervention strategies and respective feature sets of the plurality.

In some embodiments, the intervention action is part of a sleep intervention strategy that includes a plurality of intervention actions. For example, the instructions can be executable to communicate the message that is indicative of the sleep intervention strategy and including an order of the plurality of intervention actions, and to revise the predictive data model including revising one or more of the order of the plurality of intervention actions and the plurality of intervention actions.

Various embodiments are directed to a system comprising input circuitry, memory circuitry, and processing circuitry. The input circuitry is to receive data indicative of a current psychophysiological state of a user. The memory circuitry is to store a predictive data model indicative of different patterns and probabilities of the user transitioning from an awake state to a sleep state. The processing circuitry is to: detect, using the data, a pattern among the different patterns of the predictive data model that is indicative of a probability of the user transitioning from the awake state to the sleep state at a date and time; based on the detected pattern, identify a sleep intervention strategy including at least one intervention action predicted to increase the probability of the user transitioning to the sleep state at the date and time; and communicate at least one message indicative of the at least one intervention action to the user.

In some embodiments, the memory circuitry includes instructions that when executed cause the processing circuitry to generate the predictive data model based on general population trends and publically available information, and to revise the predictive data model for the user over time using feedback data indicative of success of different sleep intervention strategies for respective feature sets.

In some embodiments, the at least one message indicative of the at least one intervention action further includes an indication of an order and timing of the at least one intervention action. For example, the processing circuitry can communicate the at least one message to another device to automatically cause the at least one intervention action to occur at a particular time in accordance with the sleep intervention strategy.

Embodiments in accordance with the present disclosure include all combinations of the recited particular embodiments. Further embodiments and the full scope of applicability of the invention will become apparent from the detailed description provided hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description. All publications, patents, and patent applications cited herein, including citations therein, are hereby incorporated by reference in their entirety for all purposes.

### BRIEF DESCRIPTION OF THE DRAWINGS

Various example embodiments may be more completely understood in consideration of the following detailed description in connection with the accompanying drawings, in which:

FIG. 1 illustrates an example of a system for sleep management, in accordance with various embodiments;

FIG. 2 illustrates an example computing device including non-transitory computer-readable medium storing executable instructions, in accordance with the present disclosure;

FIG. 3 illustrates another example system for sleep management, in accordance with various embodiments;

FIG. 4 illustrates another example sleep management system, in accordance with various embodiments;

FIG. 5 illustrates an example method for data processing by the sleep management system of FIG. 4, in accordance with various embodiments;

FIG. 6 illustrates an example sleep intervention strategy, in accordance with various embodiments;

FIG. 7 illustrates an example process for generating a predictive data model, in accordance with various embodiments;

FIGS. 8A-8C illustrate different examples of predictive data models, in accordance with various embodiments;

FIG. 9 shows a sample effect of the intervention on heartrate (HR), heartrate variability (HRV) over time, and the use of an intervention planner to generate sleep intervention strategies for a user, in accordance with various embodiments;

FIG. 10 shows inter-beat interval times for one falling asleep with and without intervention (in this case, virtual reality biofeedback), in accordance with various embodiments;

FIGS. 11A-11B show group level relationship between perceived pre-sleep cognitive arousal and subsequent physiological time spent falling asleep for those with and without insomnia, in accordance with various embodiments;

FIG. 12 shows a relation between physiological pre-sleep state of activation (cortisol levels) and night-time polysomnographic sleep efficiency, in accordance with various embodiments;

FIG. 13 shows a diagram of pre-sleep effect on sleep, in accordance with various embodiments; and

FIG. 14 illustrates a theoretical plot of the relationship between pre-sleep physiological autonomic activation (e.g., HRV), and subsequent nighttime sleep efficiency, in accordance with various embodiments.

While various embodiments discussed herein are amenable to modifications and alternative forms, aspects thereof have been shown by way of example in the drawings and will be described in detail. It should be understood, however, that the intention is not to limit the invention to the particular embodiments described. On the contrary, the intention is to cover all modifications, equivalents, and alternatives falling within the scope of the disclosure including aspects defined in the claims. In addition, the term “example” as used throughout this application is only by way of illustration, and not limitation.

### DETAILED DESCRIPTION

Aspects of the present disclosure are believed to be applicable to a variety of systems and methods involving a sleep management system that can track sleep states over time, and identify what biological, individual, and environmental factors cause difficulty with transitioning from an awake state to a sleep state for a user. In specific embodiments, the system can communicate an intervention action to the user to increase a probability of transitioning to the sleep state and is updated over time based on feedback data indicative of success or failure of intervention actions. While the present invention is not necessarily limited to such applications, various aspects of the invention may be appreciated through a discussion of various examples using this context.

Accordingly, in the following description various specific details are set forth to describe specific examples presented herein. It should be apparent to one skilled in the art, however, that one or more other examples and/or variations of these examples may be practiced without all the specific details given below. In other instances, well known features have not been described in detail so as not to obscure the description of the examples herein. For ease of illustration, the same reference numerals may be used in different diagrams to refer to the same elements or additional instances of the same element.

Embodiments in accordance with the present disclosure involve a system for improving sleep in users by optimizing a psychophysiological state of a user at bedtime or other times when attempting to sleep (e.g., daytime nap). Such embodiments are adaptable and personable to the user. Embodiments are directed to lowering hyperarousal (e.g., altered state of psychophysiological activation) which prevents the user from falling asleep and obtaining a restful night. The system can use data indicative of a current psychophysiological state of the user to detect a pattern of a predictive data model that is indicative of the probability of the user transitioning from an awake state to a sleep state at a data and time, and based on the detected pattern, select an intervention action predicted to increase the probability of the user transitioning to the sleep state at the date and time. The intervention action can include or be associated with a sleep intervention strategy to increase or improve sleep for the user. Improving sleep can include improving falling asleep (e.g., smoother physiological changes in the transition from wake-to-sleep), improving sleep processes (e.g., reducing the arousal threshold, increase sleep efficiency, enhance nighttime cardiovascular function) and/or reducing the transition time from wake-to-sleep (e.g., faster perceived and/or physiological sleep onset latencies). In some embodiments, the sleep intervention strategy is a pre-sleep intervention strategy. In various embodiments, the strategy can be across the whole wake-to-sleep transition, such as not being limited to pre-sleep. The system can communicate a message indicative of the intervention action to the user, such as directly to the user or to another device to perform the intervention action or communicate with the user.

In some embodiments, the predictive data model can be associated with a plurality of different patterns. Each of the patterns include or are associated with a feature set of different factors that can impact the psychophysiological state of the user, an order and timing of the factors occurring, among other attributes of the factors, such as amplitude or strength. In some embodiments, the factors can impact a transition time from an awake state to a sleep state transition time, herein generally referred to as “a transition time”. The patterns can be indicative of different probabilities of the user transitioning to the sleep state at a particular date and time and/or different intervention actions which can improve the probability and/or improve the transition time. In some embodiments, the different patterns are associated with different feature sets and the detected pattern is used to select a sub-model of the predictive data model. For example, the data is input to the predictive data model, which uses the input data to identify the pattern (e.g., a feature set among a plurality of feature sets) and output a probability of the user transitioning to the sleep state. In some embodiments, the output of the predictive data model includes an intervention action to improve the probability sleep and/or transition time to sleep. In addition or alternatively, the intervention action can be used to manipulate an acute psychophysiological state of the user, such as improving relaxation or reducing sympathetic activation. In other embodiments, the output is used to select the intervention action.

As used herein, “hyperarousal” includes or refers to an abnormal state of psychophysiological activation, which can be reflected across different biological domains (e.g., elevated heart rate, high cortisol levels, elevated high-frequency EEG activity), psychological domains (e.g., worry, concerns, rumination), and emotional domains, and can be the result of exaggerated responses to stimuli (e.g., hyperarousal can be induced by stress, by the fear of not able to sleep, and/or triggered by financial concerns, etc.). Hyperarousal can be used to describe a state of being that aggravates a user such that a transition time from an awake state to an asleep state is prolonged or prevented when a user is not relaxed or is worried.

“Awake state”, as used herein, includes or refers to being in a state of awareness of one's surroundings; where a person's consciousness is preserved. Biologically, an awake state can be characterized by a desynchronized fast and low amplitude EEG activity. “Sleep state”, as used herein, includes or refers to being in a state of unconsciousness of one's surroundings with reduced responsiveness to environmental stimuli. Biologically, sleep is accompanied by a progressive increase in cortical synchronization, and a general state of physiological deactivation (e.g., reduction in metabolism).

Embodiments in accordance with the present disclosure are directed to targeting factors preventing users from falling asleep and having a good sleep, such factors can cause bedtime hyperarousal. Hyperarousal can manifest as heart racing, muscle tension, worry about sleep, as well as other non-sleep related concerns such as mental hyper-activation, racing thoughts, pain-related discomfort and anxiety. Hyperarousal manifests differently in different people, has different causes, and involves different domains as noted above.

In some embodiments, different intervention action strategies can be used to target specific aspects of hyperarousal. In many instances, different users have unique sleep and relaxation needs, and these needs, as well as factors affecting stress and sleep, change over time. For example, users have different personalities, respond differently to external stimuli, are immersed in different socioeconomic environments, and can require individualized sleep promoting solutions. Embodiments of the disclosure are directed to a predictive data model which is used to recognize that users have different preferences for relaxation techniques and sleep solutions, and that similar solutions have differing efficacy on different users.

The predictive data model can be adaptive and personalized to assist user relaxation and sleep by focusing on the transition from an awake state to a sleep state. For example, the predictive data model can be used to reduce the transition time from the awake state to the sleep state by reducing the pre-sleep arousal state. Various embodiments target the psychophysiological state across the falling asleep process, a time window in which the user uses to reach a specific psychophysiological state to allow the transition from wake-to-sleep. The predictive data model is adaptable to users and over time by identifying different factors (e.g., psychophysiological stress, consumption of stimulants or other substances, exaggerated physical activity in the evening, pain) responsible for bedtime psychophysiological arousal, and recognizing the factors vary within and between users. The predictive data model can recognize that different single intervention actions or multiple intervention actions targeting pre-sleep hyperarousal (e.g., cognitive-behavioral interventions, relaxation strategies, virtual reality immersion, neuromodulation, brainwave entrainment) can have different efficacy in reducing the bed-time hyperarousal, with efficacy varying among users and with time. For example, users have different preferences on how to relax and these preferences interfere with an effectiveness of intervention actions. Further, different strategies or combinations can have different instantaneous effects (e.g., minute resolutions) and impacts can vary across different days, months, etc.

The system, in various embodiments, can be used to dynamically sense data indicative of physiology (e.g., HR via a plethysmography sensor) and behaviors (e.g., amount of daily physical activity), environmental conditions (e.g., humidity, temperature, light), and deliver a multicomponent intervention (e.g., cognitive and behavioral interventions, meditation, relaxing virtual immersion, direct current stimulation, relaxing audio) via one or multiple actuators (e.g., speaker, virtual reality headsets, drug patches) to relieve stress, promote, and sustain sleep based on sleep and relaxation needs of a user. The system includes a predictive data model which can include machine learning (ML) and/or artificial intelligence (AI) techniques to optimize the intervention in real-time for the user, considering multiple features, such as the psychophysiological state of the user and the effectiveness of a particular sleep intervention strategy at any particular point in time. The system and the predictive data model can: 1) target a factor, e.g., hyperarousal, during a time-window, e.g., sleep onset period, sometimes herein referred to as the “transition time”, to falling asleep and maintain sleep; 2) implement AI and/or ML to reach a highly adaptive and personalized single or multicomponent intervention to maintain effectiveness and user engagement over time.

The systems and methods of implementing the system can view timing, specificity, adaptability, and personalization to allow for the optimization of the psychophysiological state of the user in the pre-sleep period. Due to the diversity and constant dynamic modification of the features contributing to poor sleep and stress, the preference of the user and the effectiveness of different treatment solutions for the user, the system can adjust intervention actions for efficacy, generalizability, and sustainability. In addition, timing and specificity of the intervention actions can be adjusted.

Specific embodiments are directed a system for managing and/or improving sleep. In some embodiments, the system is an individualized sleep management system that aids with minimizing the transition time from an awake state to an asleep state for a user. However, embodiments are not so limited and the system can improve sleep in a variety of ways, such as minimizing the transition time, improving how the user falls asleep, improving continuity measures, etc. The system gathers data about a user, analyzes the collected data, and, based upon that data and a predictive data model, selects intervention actions(s) to minimize the transition time between awake to asleep states for the user. The system can gather data about a user including data about which mitigating intervention strategies have worked best in previous similar instances and present the strategy plan to the user. In some embodiments, the user can provide feedback to the system as to which intervention(s) worked under a given set of conditions. The sleep management system can provide external action intervention based upon data input as well as drawing upon prior intervention sessions to arrive at a sleep intervention strategy for a current psychophysiological state of the user prior to sleep.

Various embodiments are directed to a system including input circuitry (e.g., sensor or other types of circuitry), memory circuitry, and processing circuitry. The input circuity can obtain current physical measurements associated with a user and use the physical measurements either by themselves or in conjunction with other data to provide the user with an intervention strategy for that day. The physical measurements obtained by the input circuitry can include one or more physiological signals, environmental information, and so on. In some embodiments, a wearable device is associated with the sleep management system, where the wearable device is able to track a state of the user throughout a day to aid with devising a plan for minimizing transition time from awake to asleep for the user.

In some embodiments, the processing circuitry generates the predictive data model to minimize the transition time between the awake state to the sleep state for individual users. The processing circuitry can update and revise the suggested sleep intervention strategy based upon daily tasks, stressors, physiological measurements, and user inputs. The user inputs can include mood, items consumed, planned interactions with others, and so forth. In some instances, the processor circuitry can synchronize with a daily calendar of the user as a starting point for building a sleep intervention strategy for when the user is ready to sleep. The processing circuitry can trigger a plurality of intervention actions based upon data input, data detected or measured, and data obtained through ML. For example, the sleep management system can remind a user that a mediation session prior to trying to sleep can be useful based upon the calendar of the user for that day. Another example includes the sleep management system reminding a user to have a cup of warm tea one and a half hours before attempting to sleep based upon the meals input by the user for that particular day.

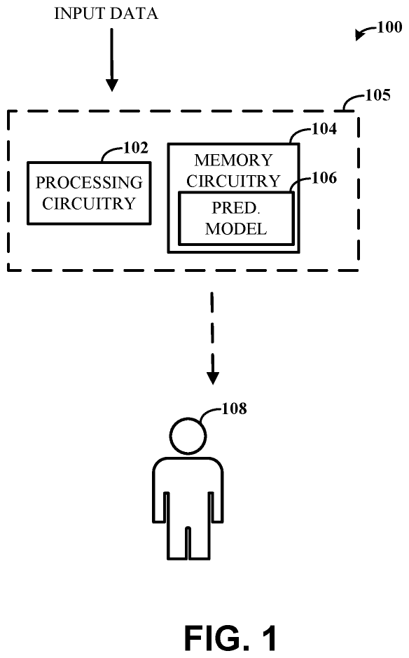

Turning now to the figures, FIG. 1 illustrates an example of a system for sleep management, in accordance with various embodiments. The system 100 can be used for managing transitions from an awake to a sleep state for a user 108. For example, the system 100 can optimize the psychophysiological state of the user 108 across the falling asleep process, the time window in which the user 108 uses to reach a specific psychophysiological state to allow the transition from wake to sleep and/or to improve sleep.

The system 100 include processor circuitry 102 and memory circuitry 104. In some embodiments, the processor circuitry 102 and memory circuitry 104 form part a device 105, such as being logic circuitry of the device 105. In other embodiments, the processor circuitry 102 and memory circuitry 104 form part of separate devices that communicate with one another, such as with distributed computing devices across a network. The memory circuitry 104 can store a predictive data model 106. In some embodiments, the memory circuitry 104 further stores instructions executable by the processing circuitry 102 to cause the below described processing. The predictive data model 106 can be indicative of different patterns and probabilities of the user 108 transitioning from an awake state to a sleep state. In some embodiments, the predictive data model 106 can be associated with an intervention action to increase the probability of the transition and/or decrease the transition time at the date and time, as further described herein. In some embodiments, the different patterns include or are associated with different features sets. For example, the patterns can include sleep patterns for the user which are associated with the different feature sets. The different patterns can be indicative of different probabilities of the user 108 transitioning from an awake state to a sleep state at a time and date based on the predictive data model 106 and the feature set and/or indicative of different intervention actions that can increase the probability of the transition and/or increase a probability of decreasing the transition time.

Features, as used herein, include factors which can impact a psychophysiological state of the user 108, an order and/or timing of the factors, a current time and/or date, and other attributes of the factors. Such factors can, for example, impact sleep for the particular user 108, and/or for a plurality of users (e.g., in general, based on demographics, etc.) As further described herein, some factors can negatively impact sleep, such as negatively impacting (e.g., increasing) the transition time between the awake state to the sleep state for the user 108. Some factors can positively impact sleep, such as positively impacting (e.g., decreasing) the transition time. For example, the feature sets can be associated with factors that prevent people from falling asleep, such as those that can cause hyperarousal around bedtime for a user. Hyperarousal can manifest as heart racing, muscle tension, stress or worrying, as well as other manifestations, such as mental hyperaction, racing thoughts, pain-related discomfort, and anxiety. Hyperarousal can manifest differently in different users, have different causes (e.g., factors), and involve different domains (e.g., cognitive, emotional, physiologic).

In some embodiments, the predictive data model 106 can include a plurality of sub-models. The plurality of sub-models are associated with the plurality of patterns (e.g., the feature sets). Each pattern can be associated with at least one sub-model. In some embodiments, multiple sub-models of the plurality can be associated with a respective pattern, such as each pattern being associated with multiple sub-models. As an example, each of the multiple sub-models for the respective pattern can be associated with a different time frame (e.g., millisecond, seconds, minutes, hours, days, weeks, months) and the multiple sub-models can be concurrently running. The plurality of sub-models can each indicate the probability of the user transitioning from an awake state to a sleep state at particular times of the day and/or with different transition times in response to different intervention actions.

Each of the sub-models can be associated with a particular feature set of the plurality of feature sets, with each feature of the particular feature set having a weight. The weight can be based on or indicative of how predictive the respective feature is for the user to or not to transition to the sleep state and/or the impact of the feature on the transition time. In some embodiments, respective intervention actions can increase the probability of transitioning to a sleep state and/or decrease the transition time differently for different sub-models and associated feature sets (e.g., factors impacting sleep for a time frame). As previously described, the predictive data model 106 can be used to predict transitioning to the sleep state based on the intervention action, and to improve sleep cycles and transition times over time for the user 108.

The processing circuitry 102 can use the data indicative of the current psychophysiological state of the user 108 to detect a pattern among the different patterns of the predictive data model 106 that is indicative of the probability of the user 108 transitioning from the awake state to the sleep state at the date and time. The processing circuitry 102 can process the data to identify the feature set from the data received and to detect the pattern of features using the feature set.

The predictive data model 106 can include an AI model or machine learning model (MLM). Various ML frameworks are available from multiple providers which provide open-source ML datasets and tools to enable developers to design, train, validate, and deploy MLMs, such as AI/ML processors. AI/ML processors (also sometimes referred to as hardware accelerators (MLAs), or Neural Processing Units (NPUs)) can accelerate processing of MLMs. ML processors are integrated circuits (ASICs) that can have multi-core designs and employ precision processing with optimized dataflow architectures and memory use to accelerate calculation and increase computational throughput when processing MLMs.

For example, the predictive data model 106 can receive the input data, identify the pattern from the input data, and based on the pattern, output a probability of the user transitioning to the sleep state. In some embodiments, the output can be used to select the intervention action to increase the probability of transition and/or decrease the transition time to the sleep state. In some embodiments, the output includes the intervention action. For example, the output can include the probability of the user transitioning to the sleep state in response to the intervention action.

Example data indicative of the current psychophysiological state of the user includes factors such as a physiological measurement and a pattern of day, such as a routine, consumption data, environment data, reproductive information, among other data. For example, the physiological measurements can include, but are not limited to, HR, HRV, breathing rate, muscle tone, body temperature, forehead temperature, and skin conductance. Other example data includes circadian rhythm, user reproductive stage (e.g., puberty, menstrual cycle phase, menopause), personality traits (e.g., introvert, narcissistic, depressed), medical conditions, and demographics (e.g., age, sex, race, type of job), among others. The environment data can include light intensity, noise, temperature, humidity levels, and pollen levels, among other environmental factors. Consumption data can include food, liquid, caffeine, and other drug consumption (e.g., nicotine, prescription, medical cannabis, non-prescription medication and substances), the amount consumed, and time when consumed. Exercise data can include a type, time, and/or intensity of the exercise. Other user behaviors or routine can be input, such as sexual activity, relaxation activity (e.g., meditating, reading), television or other media watched (e.g., reading on a computer, watching a movie), and stressful activity (e.g., job interview, traveling, driving in traffic, political events, social events). In some embodiments, activities which are relaxing and which are stressful can be identified by the user 108 (e.g., the user manually labels) and/or using the predictive data model 106. For example, the data input can include user preferences, such as identified intervention actions (e.g., guided mediation verses breathing awareness, yoga, spiritual verses non-spiritual, and music types).

The data can be input from a variety of sources. In some embodiments, the source can include a separate device, such as sensor circuitry, third party platforms, mobile application, and web platforms. The data can be general to a population of users or specific to a user. For example, the data general to a population of users can include information based on clinical studies, endorsements, and journals. Such data can be used to generate the predictive data model 106 and/or as feedback data. For example, scientific data can include a relevance of features and targeted intervention plans that is scientifically informed, which may result from scientific studies that include clinical trials. The data can be automatically provided or manually input by the user 108. For example, calendar data, geolocation data, perceived alertness, exercise data, consumption data, emotional state, and user preferences can be obtains from other applications, sensors, and/or self-entered by the user 108 to a user interface (UI).

In some embodiments, the processing circuitry 102 can detect the pattern by identifying the feature set from the plurality of feature sets from the input data and selecting the sub-model from the plurality of sub-models of the predictive data model 106 based on the identified feature set. As described above, the plurality of sub-models can be indicative of the probability of the user 108 transitioning to the sleep state in response to different intervention actions, and associated with a particular feature set of the plurality of feature sets. In some embodiments, the plurality of sub-models include sub-sets of sub-models for each pattern which are associated with different time frames including a short time frame, a mid-time frame, and a long-time frame, and each feature of the set can have a weight associated with the probability of the user transitioning to the sleep state and/or an impact on the transition time (e.g., associate different feature sets and time frames).

The processing circuitry 102, based on the detected pattern, can select an intervention action predicted to increase the probability of the user transitioning to the sleep state at the data and time and/or to otherwise improve sleep, such as improving the falling asleep and/or sleep processes. The selection can include an output of or be based on the predictive data model 106. The intervention action can be part of a sleep intervention strategy that includes the intervention action and/or a plurality of intervention actions. Example intervention actions include a behavioral intervention action, a cognitive intervention action, a neuromodulation action, an environmental change, a sensory action, and combinations thereof. The intervention action can be passively instructed to the user 108 to perform or actively instructed to another device to perform (e.g., turning on music, lower lights, decreasing the temperature, among others). Example intervention actions are further described herein. The intervention action can be used to target a different hyperarousal state.

The processing circuitry 102 can communicate a message indicative of the intervention action to the user 108. The message can be communicated directly to the user 108, such as via a UI (user interface) associated with the processing circuitry 102. In other embodiments, the processing circuitry 102 can communicate the message to another device to perform the intervention action or to communicate the message to the user 108.

In some embodiments, the processing circuitry 102 can communicate a message or plurality of messages associated with a plurality of intervention actions that includes the intervention action. For example, the processing circuitry 102 can communicate the message that is indicative of a sleep intervention strategy and which includes an order of the plurality of intervention actions. In some embodiments, the processing circuitry 102 communicates a plurality of messages, including the message, that are indicative of the plurality of intervention actions, each of the plurality of messages being selected from the group consisting of: a message displayed to the user that instructs the user to take a respective intervention action, and a message to another device to automatically cause a respective intervention action to occur at a particular time in accordance with the sleep intervention strategy.

The intervention action and/or sleep intervention strategy can be different for different users. For example, different strategies can be used to target specific aspects of hyperarousal. Each user can have different sleep and relaxation needs, as well as factors that affect sleep and which can change for the particular user over time. Further, different users have different personalities, are in different environments, and can respond to different intervention actions (e.g., external stimuli) differently. For example, particular users can have different preferences for relaxation techniques and sleep solutions, and that similar solutions have differing efficacy. The predictive data model 106 can be informed of such preferences and/or learn the preferences over time using feedback data, as further described herein.

In some embodiments, the processing circuitry 102 generates the predictive data model 106 and stores the predictive data model 106 in the memory circuitry 104. In other examples, the processing circuitry 102 receives the predictive data model 106 and stores the predictive data model 106 in the memory circuitry 104. As previously described, the predictive data model 106 can include a MLM which is trained using input from a dataset.

Generating the predictive data model 106 can include receiving input data including factors (e.g., which are used to form feature sets) impacting the transitioning to the sleep state for the user 108 or a plurality of users, receiving known outputs associated with the inputs, identifying different patterns indicative transitioning to the sleep state (or not) within the input data, and, based on the patterns, identifying predictive probabilities of the user 108 transitioning to a sleep state at dates and times using additionally received input data. The known outputs can include past sleep transition times, time of the day, and other information. In some embodiments, the known outputs can include intervention actions which occur to increase the probability of transitioning to the sleep state and/or decrease the transition time, and which can be used to identify future intervention actions. In some embodiments, the intervention actions may otherwise or additional improve sleep for the user, such as by providing smoother physiological changes in the wake-to-sleep transition, reducing the arousal threshold, increasing sleep efficiency, and/or enhancing night-time cardiovascular function. The processing circuitry 102 receives the input and known outputs, and uses the same to generate the predictive data model 106. The input and known outputs can be actively input or passively input or received. The inputs and known outputs can include reported sleep cycles, schedule or calendar data, lifestyle data including but not limited to stress level, mood, exercise data, sleep data, and dietary data, and health information including but not limited to physiological signals or parameters, medications, diagnosis, and other treatments, as well as various combinations thereof.

The predictive data model 106 can be dynamically updated over time. For example, the processing circuitry 102 can revise the predictive data model 106 based on feedback data which is indicative of whether the user 108 transitions to the sleep state responsive to the intervention action. Such feedback data can include identification of a transition or not to the sleep state at a particular date and time, a transition time, and features occurring during the transition (such as features that occur when the user transitions and/or intervention actions). Other feedback data can include sensor data indicative of physical measurements from the user 108, user self-reports on quality of sleep, among other data. The processing circuitry 102 can receive the feedback data, and in response to the received feedback data, identify features from the feedback data, and identify whether the user 108 exhibits a response to the intervention action that is anticipated by the predictive data model 106 to increase the probability based on the identified features. In response to an unexpected response, the processing circuitry 102 can revise the predictive data model 106 for the user 108 and as associated with the detected pattern.

In some embodiments, the processing circuitry 102 can receive the feedback data in real time. In response to the feedback data and the revised predictive data model 106, the processing circuitry 102 can communicate another message indicative of a revised intervention action. In this manner, the system 100 can adaptively adjust the sleep intervention strategy for the user 108 in real time. For example, the adjusted sleep intervention strategy can include a revised intervention action that is applied during a currently-occurring sleep session (e.g., adjust in real-time) and/or for a future sleep session.

In some embodiments, the feedback data is entered by the user 108 and identifies changes in sleep cycles over time and/or user report on quality of sleep. The update can include adjusting the weights of different features based on the experienced sleep pattern and/or transition to the sleep state. As a specific example, over time, the user 108 can experience a change in sleep cycles that results in difficulty transitioning to a sleep state that is triggered by different features. In another example and/or in addition, the feedback data can be indicative of specific information of a current sleep state. As may be appreciated, different users may experience relief and/or increased ease transitioning to a sleep state using different intervention actions. The system 100 can learn which sleep intervention strategy, such as an intervention action or a series of intervention actions, at a given time and/or responsive to different feature sets, is the most effective at relaxing the user 108 and improving sleep. As the strategy is dependent on the current feature set, the system 100 can deliver the most effective strategy on a day-to-day basis. The system 100 uses data from multiple sources, at the individual or population-based level, and on different time-scales.

As a specific example, the system 100 can learn that every time the user 108 has a scheduled meeting early the next morning (calendar source) and a current pre-sleep arousal level exceeding a certain threshold (e.g., resting HR 6 bpm above baseline), the best sleep intervention strategy is to perform five minutes of respiratory bio-feedback followed by ten minutes of progressive muscle relaxation. Over time, the system 100 can learn that when, under the same circumstances, the user 108 does not exercise in the evening of that day (source from a third party platform such as a wearable activity-tracker or from other wearable devices having the disclosed system incorporate or partially integrated into the wearable device), the five minutes of respiratory bio-feedback are not necessary and ten minute of progressive muscle relaxation the outcomes of the system 100 results in the same level of efficacy.

As previously described, the processing circuitry 102 can communicate data indicative of the intervention action. For example, the processing circuitry 102 can communicate a message to the user 108 to take the intervention action to increase a probability and/or to otherwise cause transitioning to a sleep state. The intervention action can be based on past user response to an intervention action by the system 100 and/or responses of other users, such as feedback data. In other embodiments and/or in addition, the intervention action includes a computer-readable instruction that is communicated to another device, such as to sensor circuitry, to temperature control circuitry (e.g., associated with a heating, ventilation, and air conditioning (HVAC) system), and/or light control circuitry, etc. As non-limiting examples, the intervention action can be an instruction provided to an HVAC system to change the temperature of a particular room, an instruction to a user device to provide a notification to the user, such as a smart watch beeping to notify the user to turn off devices or perform other actions, and/or a display on an application executed by a smartphone which instructs the user on a particular action to take, among other specific actions. As may be appreciated, embodiments are not limited to a single action and multiple intervention actions may be triggered by the system 100. Other example intervention actions can include suggestions to the user or actions for stress relief, which can increase a probability of transitioning to the sleep state or otherwise improve sleep for the user. Example stress relief strategies can include cognitive behavior therapy, music therapy (e.g., activate a device to play music to the user or recommend the same to the user), hormonal therapy and/or aromatherapy (e.g., activate a device that outputs a scent or recommend the same to the user, such as lavender, chamomile, or rose scents). Example suggestion can include recommending the user exercise, take supplements, aromatherapy or hormonal therapy, play music, reduce caffeine intake, chew gum, and deep breathing exercises, among other recommendations.

As noted above, the system 100 can further include input circuitry. The input circuitry can receive the data indicative of the current psychophysiological state of the user. The data can include schedule or calendar data, stress level, general mood, dietary data, health information, exercise data, sleep data, and a combination thereof. In some embodiments, the input circuitry can include sensor circuitry used to obtain a physical measurement associated with a user 108. In some embodiments, the input circuitry includes a wearable physiological sensor to sense a physiological signal from the user 108 and a sensor to sense an atmospheric measurement. The input circuitry, such as the sensor circuitry, has a communication circuit for communicating the physical measurement to the processing circuitry 102. The communication circuit can communicate in a wireless or wired manner.

The physical measurement can be a physiological signal or measurement (e.g., body fluids) from the user 108, motion, and/or an atmospheric measurement from the environment surrounding or near the user 108. For example, the input circuitry can include a wearable physiological sensor, such as a wearable device, that senses the physiological signal from the user. The input circuitry can alternatively or in addition include a sensor to sense an atmospheric measurement. Example physiological signals include parameters such as blood pressure, HR, skin conductance, body temperature, etc. Example atmospheric measurements include air temperature, atmospheric pressure, humidity, etc. Although embodiments are not limited to a physiological signal or atmospheric measurement and can additionally or alternatively include motion data (e.g., from accelerometer), and/or global positioning data (GPS).

Accordingly, in some embodiments, the system 100 includes but is not limited to a UI (e.g., application, wearable device, web platform), sensors (e.g., GPS sensor, accelerometer, microphone, skin conductance, photoplethysmographic, ambient light, skin and environmental temperature sensors, pressure and chemical sensors, microphone), actuators (e.g., speaker, screen, virtual reality headsets, neuromodulators, drug patches, light) to deliver the intervention actions (e.g., cognitive-behavioral intervention, meditation strategy, neuromodulation, bio-feedback, virtual immersion, light exposure, brainwave entrainment), a connectivity module (e.g., WiFi, Bluetooth), a cloud computing system, and AI-based data processing modules.

The system 100 includes ML and AI based methods, informed by grounded science. The system 100 can increase and/or maximize the effectiveness of a single or a multicomponent intervention actions to relieve stress and improve sleep across the wake-to-sleep transition. For example, the system 100 allows for delivering a personalized intervention package, dynamically optimized based on the needs of the user, their physiological state, and other factors over time and as an individual's needs change.

The system 100 can learn which sleep intervention strategy (single intervention action or combination of intervention actions), at any given time, is most effective to relax the user 108 and improve sleep. For example, every night at bedtime the system 100 can deliver to the user 108 the most effective strategy.

The system 100 is not limited to the delivery of sleep intervention strategies directly focused at optimizing a pre-sleep psychophysiology of the user 108. The system 100 can promote behaviors of other users and/or other behaviors of the user 108, such as sleep hygiene rules as well as actuate other strategies (e.g., reinforce physical activity routine, meditation, promote positive attitudes toward life) outside the bedtime hours and/or across the complete wake-to-sleep transition.

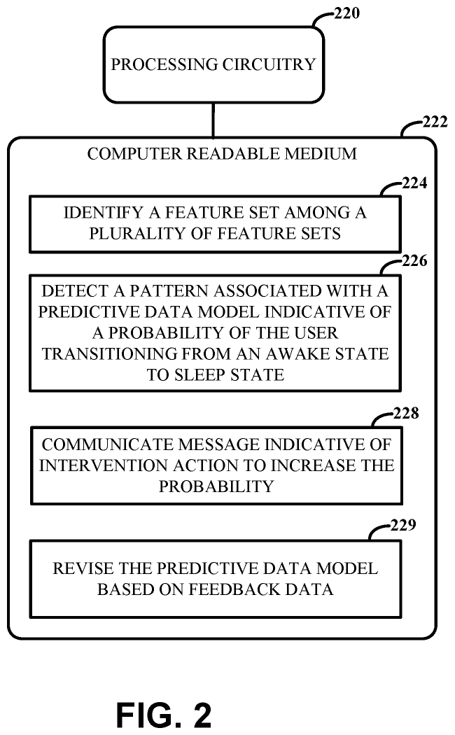

FIG. 2 illustrates an example computing device including non-transitory computer-readable medium storing executable instructions, in accordance with the present disclosure. The computing device, in accordance with examples herein, includes a user device having logic circuitry, such as the processing circuitry and memory circuitry illustrated by FIGS. 1-2.

The computing device includes processing circuitry 220 and memory circuitry. The memory circuitry includes computer readable medium 222 storing a set of executable instructions 224, 226, 228, 229. The computer readable medium 222 can, for example, include read-only memory (ROM), random-access memory (RAM), electrically erasable programmable read-only memory (EEPROM), Flash memory, a solid state drive, and/or discrete data register sets.

At 224, the processing circuitry 220 identifies a feature set among a plurality of feature sets from data, the data being indicative of a current psychophysiological state of a user. The processing circuitry 220 can receive the data indicative of the feature set

At 226, the processing circuitry 220, based on the identified feature set, detects a pattern associated with a predictive data model that is indicative of a probability of the user transitioning from an awake state to a sleep state at a date and time. Detecting the pattern can include instructions executable to identify, in the data, a feature set from a plurality of feature sets and to select a sub-model of the predictive data model using the identified feature set.

At 228, the processing circuitry 220, based on the detected pattern and the predictive data model, communicates a message to the user indicative of an intervention action predicted to increase the probability of the user transitioning to the sleep state at the date and time. At 229, the processing circuitry 220 revises the predictive data model based on feedback data which is indicative of whether the user transitions to the sleep state in response to the intervention action and/or other information on sleep, such as the transition time.

In some embodiments, the instructions are further executed to, in response to the feedback data and the revised predictive data model, communicate another message indicative of a revised intervention action. For example, the processing circuitry 220 can revise a weight provided to the intervention action in response to the identified feature set for the user in the predictive data model. In some embodiments, the processing circuitry 220 can revise the predictive data model over time for the user based on the feedback data and additionally received feedback data that is indicative of different sleep intervention strategies and feature sets (e.g., indicative of the success or failure of strategies in response to different feature sets for the user and/or generic data provided from databases).

In some embodiments, the intervention action is part of a sleep intervention strategy that includes a plurality of intervention actions. In some such embodiments, the processing circuitry 220 can communicate the message that is indicative of the sleep intervention strategy and an order of the plurality of intervention actions, and revise the predictive data model including revising the order of the plurality of intervention actions and/or the plurality of intervention actions.

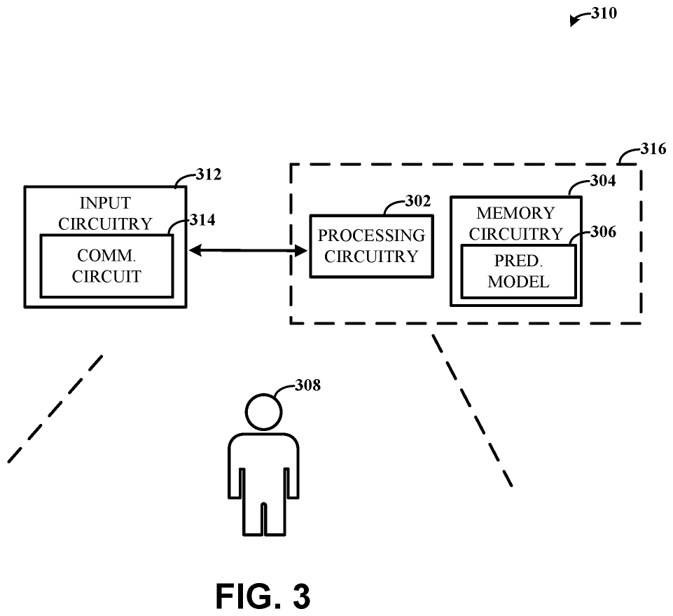

FIG. 3 illustrates another example system for sleep management, in accordance with various embodiments. The system 310 includes processing circuitry 302, and memory circuitry 304 that includes a predictive data model 306, as previously described in connection with FIG. 1, and additionally includes input circuitry 312. In some embodiments, the processor circuitry 302 and memory circuitry 304 form part a device 316, however, embodiments are not so limited. In various embodiments, the system is for acute psychophysiological state and sleep management.

The input circuitry 312 can receive the data indicative of the current psychophysiological state of the user 308, as previously described. The input circuitry 312 can include a plurality of different types of devices. As previously described, the input circuitry 312 can include sensor circuitry that obtains a physical measurement from the user 308 and communicates the physical measurement to the processing circuitry 302 using the communication circuit 314. Although the input circuitry 312 is illustrated as a single circuit, embodiments are not so limited. For example, the input circuitry 312 can include a plurality of sensors that obtain different physical measurements, such as different physiological signals, atmospheric measurements, motion data, and/or GPS signals. In a specific embodiment, the input circuitry 312 includes a wearable physiological sensor to sense a physiological signal from the user 308 and another sensor to sense an atmospheric measurement, as previously described. Further, the input circuitry 312 is not limited to sensor circuitry and can include other circuitry, such as a UI for the user 308 to input data to.

The memory circuitry 304 stores the predictive data model 306 indicative of different patterns and probabilities of the user 308 transitioning from an awake state to a sleep state and, in some embodiments, computer readable instructions. The computer readable instructions are executable by the processing circuitry 302 to perform the following.

The processing circuitry 302 can detect, using the data, a pattern among the different patterns of the predictive data model 306 that is indicative of a probability of the user 308 transitioning from the awake state to the sleep state at a date and time. As previously described, the processing circuitry 302 can process the data to identify a feature set from the data received, and to detect the pattern using the feature set. Based on the detected pattern, the processing circuitry 302 can identify a sleep intervention strategy including at least one intervention action predicted to increase the probability of the user 308 transitioning to the sleep state at the date and time and/or to otherwise improve sleep. The processing circuitry 302 further communicates at least one message indicative of the at least one intervention action to the user 308.

In some embodiments, the processing circuitry 302 generates the predictive data model 306 that indicates the probability of the user 308 transitioning to the sleep state at the date and time. The predicative data model 306 can be generated based on general population trends and publicly available information, among other data. In some embodiments, the processing circuitry 302 can revise the predictive data model 306 for the user 308 over time using feedback data indicative of success and/or failure of different sleep intervention strategies for respective feature sets.

In some embodiments, the intervention action can include a plurality of actions. For example, the processing circuitry 302 can communicate the at least one message indicative of the at least one intervention action that includes an indication of an order and timing of the at least one intervention action. In some embodiments, the processing circuitry 302 can communicate the at least one message to another device to automatically cause the at least one intervention action to occur at a particular time in accordance with the sleep intervention strategy.

The sleep management systems 100, 310 illustrated by FIGS. 1 and 3 can be configured in multiple ways. Some embodiments of the sleep management systems 100, 310 can be or form part of a computer program or application that can run on a smartphone, a tablet, a desk computer, a laptop, smart watch, exercise tracker, or other independent device. In other embodiments of the sleep management system can be a web-based program. In any of the above embodiments, the program can include the executable instructions, such as illustrated by FIG. 2.

In general, a user of the sleep management system is a single user. In other configurations of the sleep management system, one system can be used by multiple user for monitoring and storing each sleep patterns for the respective user.

The systems 100, 310 described herein can use several single or multicomponent interventions to meet sleep and/or relaxation needs and user preferences for relaxation strategies. The systems 100, 310 can use or include multiple sources of data. For example, the sources can include (e.g., user physiological state, habit, behavior, environmental condition) with different time-frame (e.g., day-time behaviors known to influence sleep such as the amount of physical activity or caffeine consumption, real-time physiological indices such as current heart rate measures, morning self-report measures such as perceived alertness) used to drive the selection and the application of the sleep/stress intervention. Further, multiple personalized closed loops on a different time scale. The approach can be optimized by targeting a combination of different domains of hyperarousal.

The timing of the intervention action can be adjusted. The timing can target the falling asleep process and account for day-time behaviors known to influence sleep (e.g., amount of physical activity, caffeine consumption).

The systems 100, 310 can help users with sleep and relaxation needs (e.g., business persons, military personnel, individuals with insomnia, hospitalized patients). The systems 100, 310 can cover different uses: helping people sleeping at night, facilitating their daytime nap, or on a general level, helping people relax. Targeting stress and sleep is a condition for health and wellness. Technology adoption from users, self-reported and physiological data showing the population efficacy of the system, results from clinical trials, scientific recognition, consumer reports, and clinical endorsement for the use of this technology are among the instruments that can allow for quantifying the impact of the intervention action.

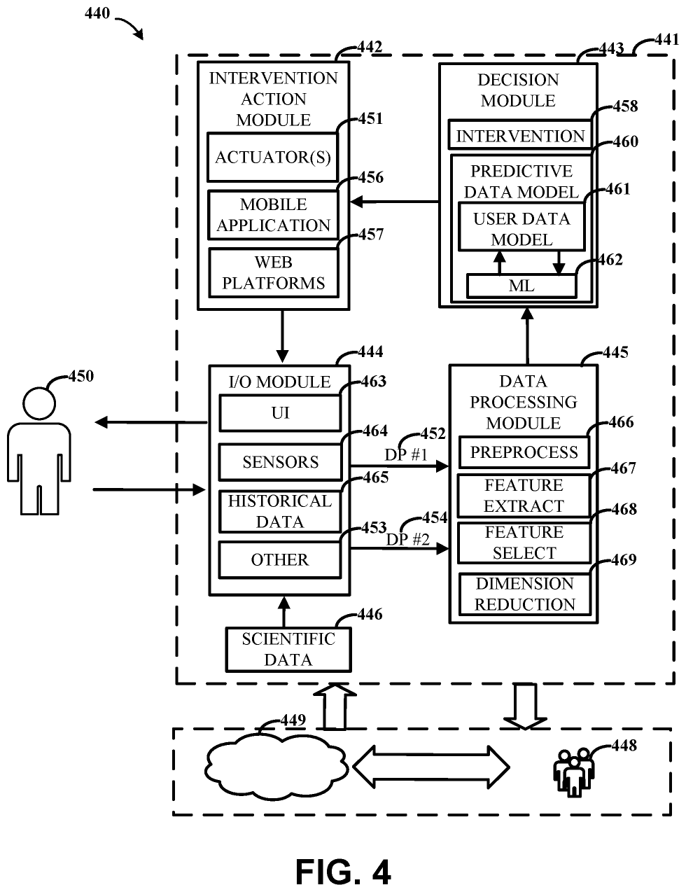

FIG. 4 illustrates another example sleep management system, in accordance with various embodiments. More specifically, FIG. 4 illustrates a functional block diagram of a sleep management system 440 that aids a user in the management of sleep cycles.

In some embodiments and as shown in FIG. 4, the sleep management system 440 includes a user module 441 that includes a data input/output (I/O) module 444, a data processing module 445, a decision module 443, and an intervention action module 442. Although embodiments are not so limited and the sleep management system 440 can include fewer or more modules than illustrated by FIG. 4. Each of the modules 442, 443, 444, 445 includes computer executable instructions which can be stored on one or a plurality of non-transitory computer-readable medium and executed by processing circuitry, such a single computing device or distribution across multiple computing devices.

As further described below, sleep and relaxation of a user 450 can be promoted by activating the intervention action module 442 as triggered by the decision module 443 receiving processed information from the I/O module 444 and informed by scientific data 446 (e.g., scientific evidence for factors affecting psychophysiological state and sleep). The decision module 443 can receive feedback data from the I/O module 444 (e.g., psychophysiological state post intervention, transition time, sleep quality).

In some embodiments, data from single and multiple users can be stored in a cloud computing system 449, which can interact with the user module 441 to optimize system performance (e.g., improve the effectiveness of the system based on collected information about outcomes stratified by demography, such as age and sex, geography of the users). The user module 441 can run independently from the data contained within the cloud computing system 449, with parameters set based on a local storage from the last update from the data stored within the cloud computing system 449.

The user module 441 can incorporate online learning functions to learn and derive predictive data models between input data and the intervention actions to be taken. In some embodiments, no data may be stored on the device of the user 450. In some embodiments, the device of the user 450 can act as a local cache of the latest user based optimized parameters. All the data, in some embodiments, can be uploaded and stored in the cloud computing system 449. This data can be used to find the most appropriate selection of the input features that have the most effective impact on sleep for the user 450, to obtain data models between the input data and the appropriate intervention actions to be taken using batch learning functionalities, and to update the predictive data model 460 frequently in accordance to the cloud derived models. The cloud computing system 449 can use data from a population of users 448 to build predictive data models to be used as an initial data model for the new users.

The I/O module 444 allows a user 450 and/or input circuitry to interface with the other modules integrated into and/or that form part of the sleep management system 440. The I/O module 444 can accept input data from the user 450 and other devices. Data can be accepted by other modules as well. In some embodiments, the user 450 can actively and/or passively input information into the sleep management system 440 in a variety of ways. A user 450 can manually enter data. A user 450 can verbally enter information into the sleep management system 440. A user 450 can upload health-related files to the sleep management system 440. In other embodiments and/or in addition, the user 450 can grant the sleep management system 440 access to data on other applications and/or other external locations, such as calendar data, exercise or food tracking applications.

Using the I/O module 444, the user 450 can enter personal data via input hardware (e.g., a mouse, a keyboard, a touch screen, a microphone etc.) such as but not limited to demographics, body mass index (BMI), ethnicity, age, user reproductive stage, medications, mood during a particular time of the day, anxiety level, activity level, allergies, types of food ingested, etc. The I/O module 444 can also be used to provide feedback data, such as the success or failure of intervention actions. In general, the I/O module 444 can be used to obtain data from and provide communications to the user 450. The system 440 can elicit responses or prompt the user 450 to perform a certain action by displaying a message and requesting a response that is entered through a UI. The responses may not be typed in some embodiments. For example, the system 440 can include a speech processing module to interpret speech and parse out responses.

The I/O module 444 can receive input from a variety of sources 463, 464, 465, 453, 446. The plurality of sources can include the user 450 with the data being obtained via a UI 463, sensor circuitry 464, historical data 465, scientific data 465, amount other data sources 453.

The data processing module 445 can process data from the I/O module 444. The data processing can include, but is not limited to low pass filtering, noise reduction, feature extraction, and so forth. Extracted features are temporal and/or spectral features representing the data and its specific time pattern, variabilities and frequency content. As shown by FIG. 4, after pre-processing of the input data, at 466, features can be extracted from each set of input categories, at 468, and can be used to form the final input feature set, at 468, which can be further dimensionally reduced, at 469.

Physiological and environmental raw signals from sensor circuitry 464 and other sources in a prior time window can form an input feature set. Temporal and/or spectral features representing the signal patterns in time and frequency can be also extracted from the raw physiological and environmental signals. Temporal features can include statistical measures such as mean, variance, and higher order statistics of the input data in a time frame. Spectral features can be extracted using Fourier transform. In some embodiments, spectral features can be spectral moments, spectral power fractions, spectral power peaks, and spectral power ratios. The features can also be extracted after applying an appropriate transform that facilitates the understanding of the input pattern such as wavelet analysis. Features can include factors of a model best representing the data in a specific time window.

Depending on the physiological signals measured, different physiologically relevant features can be extracted. Examples of such features includes HR, pulse volume, pulse transit time, etc.

User behavior and different events can be extracted from the calendar or other third-party platforms as well as a UI. The events can be clustered/classified into several categories to form an input feature set. Each event or user behavior can be represented by zeros and ones over time where 1 represents the occurrence of the event and 0 otherwise. Natural language processing (NLP) techniques could be incorporated to read the events from calendar and other third-party platforms and cluster/classify the events.

Each personality trait (this information as well as information about quality of life, coping strategies, cognitive and emotional function can be gathered through smartphone application based questionnaires) and demographic information can form an element of the input feature set.

The extracted features can form a high dimensional feature vector that is fed to the decision module 443. To reduce the complexity of the decision processing and enhance the learning process, the dimension of the feature input vector can be reduced using subspace learning and dimensionality reduction functions, at 469. Linear decomposition methods such as factor analysis, principal component analysis, singular value decomposition, and independent component analysis can be used. As the input data can exhibit a high degree of nonlinearity, nonlinear dimensionality reduction methods such as kernel-based methods can be also used. On the user module 441, online versions of such statistical methods can be used while on the cloud computing system, and the batched versions can be incorporated.

As the available data increases, the dimensionality of the features can be reduced using deep learning techniques such as the auto-encoder neural network configuration.

Depending on the feature selection method, this step can be performed on a cloud computing system 449 or both on the cloud computing system 449 and the user module 441. In some embodiments, different sets of features are used to train the predictive data model 460 and select the feature set with the best performance. Because of the computational cost and time involved, this approach can be performed by the cloud computing system 449. The predictive data model 460 is then downloaded on the user software/application to be further used. Measures can be used to select the most appropriate features which can be performed both on the user module 441 and on the cloud computing system 449. Other embodiments include use of least absolute shrinkage and selection operator (LASSO) technique where any feature with a non-zero regression coefficient is selected.

The decision module 443 can accept data from the data processing module 445 and perform various types of analyses including, but not limited to, detecting a pattern in the input data and selecting an intervention action based on the detected pattern. The decision module 443 analyzes the impact of features on the probability of transitioning to the sleep state including, but not limited to, environmental circumstances (e.g., ambient temperature, humidity, season, time of day, meal composition, caffeine and/or alcohol consumption, use of medications), individual circumstances (e.g., mood, stress, anxiety, time of day, exercise, menstrual cycle patterns, calendar events), location from GPS and/or user inputs (e.g., supermarket, home, work), and physiological state (e.g., skin temperature, thermosensitivity, HR, cardiac autonomic state, such as HRB, skin blood flow, such as peripheral vasoconstriction/vasodilation).

In various embodiments, the decision module 443 can be involved in multiple data processing streams. A first data processing stream 452 can be associated with processing input data to determine a current psychophysiological state and recommended intervention action. The second data processing stream 454 can be associated with processing feedback data from implementations (e.g., success or failure) of the intervention action.

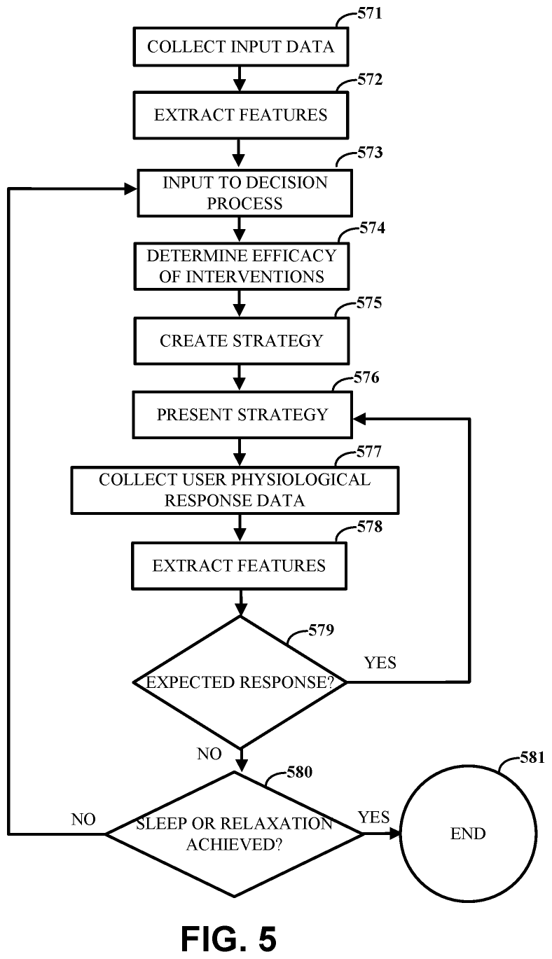

The following describes an example of the first data processing stream 452. In some embodiments, the input to the decision module 443 is a reduced set of feature best representing the input data. The outputs of the decision module 443 is a weight (or score) for every possible intervention action. Each weight can be presented from 0 to 1. These weights are processed in the intervention plan sub-module 458 to select appropriate intervention action(s) for a sleep intervention strategy. As FIG. 5 shows, the sleep management system can collect input data. The input data can be a variety of different types of data, as previously described. Once input, relevant features and correlations between the input data and effect on transition times between an awake state to asleep state can be obtained, and a decision module 443 determines the efficiency of various possible intervention actions and sequences. The decision module 443 then selects the optimal sleep intervention strategy and presents the sleep intervention strategy to the user 450 using the intervention plan sub-module 458. The sleep management system 440 can update the sleep intervention strategy throughout the day as feedback data and/or new input data is received.

The decision module 443 can include a predictive data model 460 that includes a user-specific data model 461 and ML models or functions 462 to update the user-specific data model 461 over time as new input-output relationships are revealed using feedback data and/or other revisions to the predictive data model 460. The user-specific data model 461 can be updated based on the input data and the system 440 output measures through various modalities including the UI 463, sensor circuitry 464, etc. Data from the I/O module 444 can be processed to score the intervention actions taken by the intervention action module 442 from 0 to 10, among other score ranges. The user 450 can score the intervention actions directly from 0 to 10, where 0 shows dissatisfaction and 10 shows satisfaction. Different information is extracted from the sensor circuitry 464 and can be used in accord with a scientific framework identified from the scientific data 446 to score intervention actions taken from 0 to 10. For example, the change in HR or respiration rate extracted from the sensor circuitry 464 is compared before and after the action is taken and, according to the scientific framework, is used to generate a score of 0 to 10 for each action.

The following describes an example second data processing stream 454. In some embodiments, the decision module 443 processes feedback data and generates the relevant information using scientific data 446. For example, HR and respiration rate are extracted from sensor circuitry 464 before and after the action is taken. The scores for the interventions action are added together and are scaled between 0 to 1 that is used as the target value for ML.

A simple example of the predictive data model 460 is a regression model. Another example is a neural network such as a multi-layer perceptron (MLP). Such models can be trained on-the-fly as new data is available (user module 441) and off-line as the data is uploaded to the cloud computing system 449. For example, incremental learning techniques can be used to train the network on-the-fly and batch techniques can be used on the cloud computing system 449.

The cloud computing system 449 can include similar modules as the user module 441, and may use data received from all the users 448, and it may not trigger the intervention action module 442 but rather builds a general data model that can be incorporated as the initial predictive data model for new users. The predictive data model 460 is then updated individually as the user keeps using it. The cloud computing system 449 also builds individual data models for each user based on all the data received from the user over time. Batch learning approaches can be used to derive the models.

Different approaches can be used to address missing input data. For example, users may not always accurately report time and amount of substance intake, meal, physical exercise or other inputs. As other examples, a user may not always wear the sensor device or devices collecting physiological signals and/or overall the data input can be fragmented and include sub-set of inputs needed to be reliably used by the decision module 443 to activate the intervention action module 442. In some embodiments, the missing data can be replaced by a constant, a random value, or the average of the available input samples. Other approaches such as interpolation and predictive modeling such as regression method or hidden Markov model can be also applied. Omitting the input feature set with missing data is another approach. Further, several variations of subspace ML including principle component analysis that are designed for handling the missing data problem can be used.

As previously described, inputs can contain continuous and/or discrete different sources of information on different timescales (e.g., real-time users' bio-signal, trait information such as personality). Example inputs include psychophysiological state (e.g., HR and HRV, breathing rate, muscle tone, forehead temperature, body temperature, skin conductance), circadian rhythm, reproductive stage (e.g., puberty, menstrual cycle phase, menopause), personality traits (e.g., narcissism, introversion), environment (e.g., light intensity, noise, temperature), user behaviors (e.g., time and amount of caffeine consumption, time and intensity of exercise, food intake, sexual activity, medications and substance use), stressful events (e.g., job interview, international travel, driving in traffic, political & social major events), demography (e.g., age, race, sex), user preference (e.g., guided meditation verses breathing awareness, yoga, spiritual vs. non spiritual type). The inputs can be measured using sensors and/or obtained from other sources, such as third party platforms, mobile applications, and web platforms.

In some embodiments, the data processing module 445 can accept and process data in the form of self-reports from the user, physiologic and environmental signals and/or as provided by third party platforms. The decision module 443 can integrate data inputs, and feedback data from data output and the cloud computing system 449, and from the scientific framework, to activate the intervention action module 442

In some embodiments, the inputs include or are based on scientific data 446. The scientific data 446, together with other data from the I/O module 444, inform the decision module 443. For example, the data can be based on results from scientific publications about factors affecting user stress and sleep and the effectiveness of the intervention module in reducing stress and improving user sleep. Non-limiting example awake-promoting and sleep-promoting factors are provided below in Table 1.

TABLE 1
Wake-promoting and sleep-promoting factors

| Wake-promoting factors | Sleep-promoting factors |
|---|---|
| Anxiety and stress (acting via | Alcohol (fast sleep onset) |
| activation of the stress systems | Exercise |
| hypothalamic-pituitary-adrenal axis | Circadian rhythm |
| and autonomic nervous system) | Good sleep hygiene |
| Caffeine (acting via antagonizing | Relaxation strategies (deep |
| adenosine receptors, modulated by | breathing, imagery, listening |
| timing of consumption and sensitivity | calming music) |
| to caffeine, “habitual users”) | ‘Cold temperature’ and temperature |
| Alcohol (sleep fragmentation, EEG | modulation |
| alpha intrusion, SWS suppression) | |
| Exercise (late in the evening) | |
| Circadian rhythm (falling asleep in | |
| the “wrong” circadian phase) | |
| Conditioned arousal | |
| Pain and/or body discomfort | |
| Environmental noise | |
| Engaging bedtime electronic media | |
| use (gaming, television, tablet or | |
| smart phone) | |

The intervention action module 442 can contain a set of intervention actions that can be triggered by the decision module 443. The decision module 443 can control sequence and duration of the intervention actions. Intervention actions can be singularly or simultaneously activated. Example intervention actions include behavioral intervention, cognitive intervention, environmental changes, neuromodulation, and sensory stimulation. Example behavioral intervention include breathing relaxation, breathing awareness, progressive muscle relaxation, body scan, respiratory biofeedback, guided meditation, wheel of awareness, among other interventions. Example cognitive interventions include guided cognitive exercises, identify distorted thoughts, cost benefit analysis, and thinking like a detective, among other interventions. Example environmental changes or interventions include modulated environmental lights, humidity, and temperature, among other interventions. Example neuromodulation intervention include transcutaneous vagus nerve stimulation. Example sensory interventions include brainwave entertainment, binaural beats, and binaural audio, such as playing audio, turning off a television or other device, among others. Users can select preferred actions and/or combinations to fall asleep and attributes of the actions or combination (duration, sequence, etc.) can be determined by the outcome of the decision module 443.

The intervention actions can be delivered by the intervention action module 442 using a variety of delivery channels and/or the I/O module 444. Example delivery channels include a mobile application 456, actuators 451 (e.g., audio, video, haptic stimulation), and web platforms 457. The output can contain continuous and/or discrete different sources of information on different time-scales. Different types of outputs can include the psychophysiological state of the user (e.g., HR, HRV, breathing rate, muscle tone, forehead temperature), social interactions (e.g., seeing friends, time spent on social media), mood (e.g., anxious, aggressive, apathetic), and sleep (e.g., time spent falling asleep, sleep quality, sleep duration). The output data can be measured using the sensor circuitry 464 (e.g., physiological changes), third party platforms (e.g., body weight using a weight management application), mobile applications (e.g., self-reported evaluation of sleep, emotion and cognition, results from cognitive testing), and web platforms (e.g., self-report evaluation, interview with an expert), among others.

The decision module 443 can constantly process feedback data from the I/O module 444. For example, if a “guided meditation” intervention action is active and there is no detected changes in the physiology of the user (e.g., no reduction in HR), it can be determined that the current intervention action does not physiologically relax the user 450. The decision module 443 can activate a different intervention action (e.g., breathing relaxation).

The data output can be processed in a variety of ways. The cloud computing system 449 can store system information about s single user and multiple users 448. The data can allow stratifying the effectiveness of the sleep management system according to individuals.

As described above, part from the user entered data, the system 440 can accept data from other sources including but not limited to physiologic sensors, environmental sensors, data entered into other applications that are run on the same platform etc. With regard to physiologic sensors, the sensor circuitry 464 can include but are not limited to skin conductance sensors, skin temperature sensors, blood pressure sensors, pulse rate sensors, photoplethysmogram sensors, electrocardiogram sensors, and electroencephalogram sensors. Other physiologic sensor may sense biologic signals, such as for analysis of sweat. Various physical forms of the sensors can be utilized such as but not limited to sensors that are adhesively coupled to the body, sensors that are housed in wearables and sensors that are coupled or attached to clothing.

With regard to environmental sensors, the system 440 can accept data from external sensors that are capable of communicating with the system 440 or it can accept data from sensors already integrated within the system. With an example system 440 that exists in the form of a mobile application, the sensors that already exist within the mobile device can be utilized to provide the input data into the system. Environmental data can include time of day, local temperature, local humidity, light exposure etc. Some of this data can be measured locally if sensors are utilized but in some cases, the data can be obtained from another application or an external source, such as from a website. In this case, the system can initiate a search automatically to obtain such data for the location of the user. This type of data input can be conveniently acquired if the system existed in the form of a mobile application. With regard to obtaining data from other applications, such data includes, but is not limited to, meeting data from calendar applications, or data from other menstrual management applications that can be obtained by interacting with one or multiple of these applications.

As previously described, the data processing module 445 process the data to make it more suitable for analysis. Data from each source can be processed differently. For example, processing of data from physiologic sensors includes but is not limited to low pass filtering, averaging, smoothing, and so on. Microphone data for emotionality analysis can be also similarly filtered to remove any spurious sounds. Data from websites can also be used directly. Also, sensors that produce complex waveforms such as electrocardiogram (ECG) or electroencephalogram (EEG) sensors, can be depicted by extracted parameters. For ECG, the data module 443 can extract a number of parameters such as pulse rate (PR) interval, duration of QRS complex, R-R interval etc., and store this information along with the date and time of the measured parameters.

In various embodiments, the sleep management system 440 can include or form part of a computer program or application that can run on a smartphone, a tablet, a desk computer, a laptop, smart watch, exercise tracker, or other independent device that can be contained within a wearable device or which is in data communication with a wearable device.

FIG. 5 illustrates an example method for data processing by the sleep management system of FIG. 4, in accordance with various embodiments. As described above, at 571, input data is collected, and features from the input data are extracted, at 572, such as using a data processing module. The extracted features are input to the decision process module, at 573, which can include a predictive data model. At 574, the predictive data model can be used to determine efficacy of different intervention actions and, at 575, a sleep intervention strategy is generated. At 576, the intervention action(s) of the intervention strategy is presented to the user. In response, feedback data can be collected, at 577, such as data indicative of the physiological response of the user. At 578, relevant features are extracted from the feedback data. At 579, a determination is made on whether the physiological response is expected or not. If yes, then intervention plan or strategy is continued, at 576, physiological response data is collected, at 577, and features are extracted, at 578. If the physiological response is not expected, at 580, a determination is made on whether or not sleep or relaxation is achieved. If yes, the intervention strategy is ended at 581. If not, feedback on the failure of the intervention strategy is to plug back into the data processing module, at 573, to revise the predictive data model.

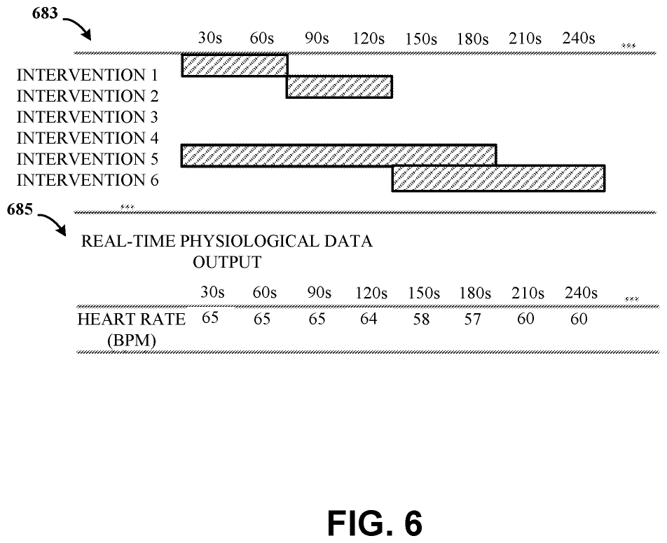

FIG. 6 illustrates an example sleep intervention strategy, in accordance with various embodiments. The following is a specific example of falling asleep when the body is active above a threshold level. It is 9:46 pm and the user wants to sleep. The input data can include: i) real-time environmental noise indicates low level noise similar to the previous three days; ii) user has a two hour meeting the next day at 8 am (from user phone calendar); iii) the user self-reported feelings of body discomfort but no particular anxiety (user input); iv) real-time HR level of the user is about 3 beats per minute (bpm) higher than the pre-sleep baseline HR of the user (averaged over the past 3 days); and v) the user performed cardio exercises in the morning between 7 am and 8 am (from wearable device linked to a smartphone of the user). The decision process module can activate an intervention action to targeted physiological arousal (e.g., breathing relaxation).

For example, FIG. 6 illustrates example intervention actions activated by the decision process module based on several levels of information (data inputs, feedback data, scientific data). The intervention actions can be activated in series (sequence) and/or in parallel (simultaneously). The intervention actions 1, 2, 4, 5 and 6 can be activated on different times and durations. In the specific example, intervention action 1 is breathing relaxation (type=behavioral . . . , propriety1=. . . , propriety2=. . . , etc.), intervention action 2 is progressive muscle relaxation (type=behavioral . . . , propriety1=. . . , propriety2=. . . , etc.), intervention action 3 is body scan (type=behavioral . . . , propriety1=. . . , propriety2=. . . , etc.), intervention action 4 is transcutaneous vagus nerve stimulation (type=neuromodulation. . . , propriety1=. . . , propriety2=. . . , etc.), intervention 5 is relaxing binaural audio (type=sensory stimulation. . . , propriety1=. . . , propriety2=. . . , etc.), and intervention 6 is respiratory bio-feedback (type=behavioral. . . , propriety1=. . . , propriety2=. . . , etc.). The top graph 683 illustrates intervention actions and different times, and the bottom graph 685 illustrates different real-time physiological output data at the different times. The different intervention actions can be subsequently chosen based on the resulting decrease in HR of the user. In the particular example, intervention action 5 can have been activated based on the user preference for a relaxing audio background. Intervention action 1 and 2 may not be successful but intervention action 6 may be successful in decreasing physiological activation.

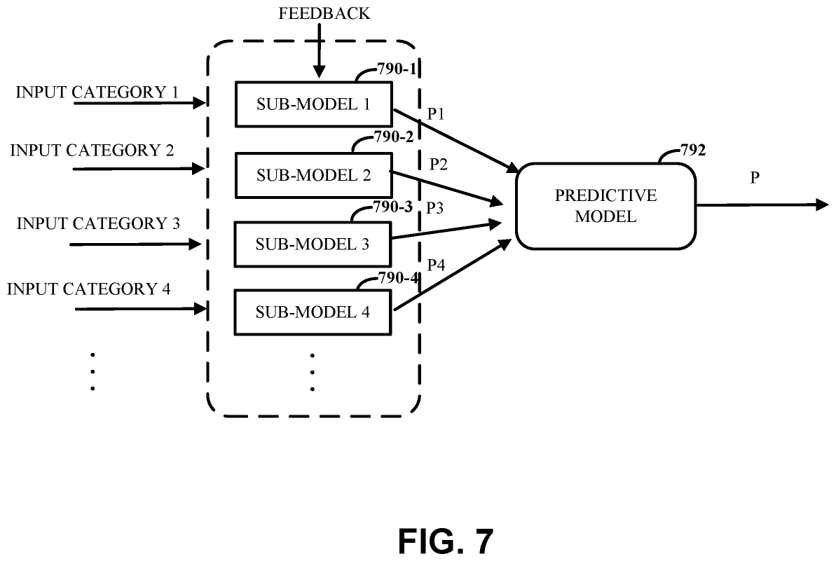

FIG. 7 illustrates an example process for generating a predictive model, in accordance with various embodiments. As described above, the decision module 443 illustrated by FIG. 4 can be executed to use and/or to generate a predictive data model 792 that indicates a probability of the user transitioning to a sleep state at a particular date and time based on input data. In some embodiments, the predictive data model 792 outputs data indicative of an intervention action that improves the probability or is predicted to cause the probability.

The predictive data model 792 accepts several different input data such as 1) physiological signals collected with non-invasive sensors and systems, 2) user routine including data extracted from the user's calendar, GPS location, etc., 3) environmental sensors including temperature, humidity, and so forth, and 4) user self-reported or input into the system, such as mood, energy level or physical state, food or drink intake over the course of the day, medication, supplements, and so forth. The predictive data model 792 can also be informed by scientific discoveries in the literature, such as health web-sites, online feeds, and/or journal articles, that can serve as an additional input. The output of the predictive data model 792 is the current and/or future probability of the user transitioning to the sleep state and which may be in response to intervention action(s). In some embodiments, the probability can be increased or is based on the intervention action occurring. A non-limiting example is where the intervention action module can be programed to communicate a message to a smart HVAC system to decrease a temperature or to a smartphone to play music.

The inputs are processed to obtain a representation that allows the decision process module to learn patterns of feature sets with respect to the transitioning or not to a sleep state for a particular user, and which can include use of different ML processes to generate sub-models 790-1, 790-, 790-3, 790-4. Each of the sub-model 790-1, 790-, 790-3, 790-4 can be associated with a different time frame for the feature set represented by input categories 1-4 and/or can be associated with different feature sets and/or output sleep intervention strategies. Different ML processes can be incorporated in the predictive data model 792 depending on the input categories of data. The ML can be used to build sub-model 790-1, 790-, 790-3, 790-4 between the inputs and the outputs which are current and/or future probabilities of sleep transition occurrence. Based on laboratory “gold standard” measure of sleep occurrence (e.g., polysomnography) and/or non-laboratory accepted sleep measures (e.g., actigraphy-based sleep/wake indication) and user self-report inputs about sleep, the feature of the predictive data model 792 are optimized by minimizing a cost function of each of the sub-models 790-1, 790-, 790-3, 790-4. The cost function is a function that maps the model predicted probability into a real number intuitively representing some “cost” associated with the predicted probability value.

The following provides example input data. The examples are not intended to be limiting and additional or fewer categories can be used.

As shown in FIG. 7, an example first input data category can include raw physiological signals or extracted features of them. Extracted features can be temporal and/or spectral features representing the physiological signals and their specific time pattern, variabilities and frequency content. Temporal features can include statistical measures such as mean, variance, and higher order statistics of the input data in a time frame. Spectral features can be extracted using Fourier transform. Spectral features in one example can be spectral moments, spectral power fractions, spectral power peaks, and spectral power ratios. The features can also be extracted after applying an appropriate transform that facilitates the understanding of the input pattern such as wavelet transform. Features could also include parameters of a model best representing the data in a specific time window. Since the dimension of the input patterns can be very high, statistical methods such as principal component analysis or linear component analysis can be used to transform the features into a lower dimension subspace where a more precise and efficient representation of the input patterns is achieved.

A second input data category can include inputs related to the user routines during a specific time such as twenty-four hours, and outputs the probability of transitioning to a sleep state at a particular time.

ML methods such as multiple regression, genetic programming, support vector regression, and difference structures of neural networks can be used for this purpose. As an example, a ML neural network with m outputs can be trained for this purpose. However, the output layer can consist of m nodes with logistic activation functions so that each output would be between 0 and 1. The cost function to be minimized can be the mean squared error and the optimization algorithm can be set to backpropagation.

The different events in the calendar can be extracted using NLP techniques and are classified or clustered into several groups according to their similarity. In an example, different events can be classified into predefined classes using algorithms such as centroid categorization, naive Bayes, etc. In another example, the events can be clustered using partitioning algorithms such as K-Means or hierarchical algorithms including agglomerative and divisive approaches.

Inputs from GPS can be used to form a similar matrix of time-location inputs. When there is a relationship between the calendar events and GPS locations and the occurrence of a sleep transition, a 3D data chart can be created where the elements represent a specific event at a specific location and on a specific time, and which can be represented by coordinates, such as illustrated by FIG. 7.

An example third input category can include environmental or atmospheric data, such as temperature, humidity, etc. and which can be treated the same way as category 1. In some embodiments, the ML process can be designed in a way that outputs the probability in several future time intervals, for example, for every 30 minutes. As an example, a neural network can be adopted. The output layer can consist of a number of outputs each representing the probability in a specific time interval in the future. The rest of the ML, optimization and cost function are the same.

An example fourth input category includes a mood of the user. The mood can be obtained every morning as a part of a self-report, e.g., by a score of 0-10. An MLP can be used to model this where the output activation function is a linear function.

The predictive data model 792 gives the probability of the transition to the sleep state occurring at a date and time based on the observed and/or collected data and/or in response to a sleep intervention strategy. A simple example is the logistic regression model which defines a linear decision boundary between the training samples associated with a sleep transition and those that are not. A more complex model can be built when there is a more complex or non-linear relationship between the inputs and output. A deep neural network such as a MLP can be used for this purpose.

To calculate the optimum network parameters (weights and biases in the case of neural networks), an optimization operation, such as backpropagation, can be used which can be done in batch or incremental style. In the batch mode, all available training data are provided to the network to calculate the optimum parameters, while in the incremental style, the parameters are updated each time a training sample is presented to the network.

The optimization of the model parameters can be done by minimizing a cost function. An example of the cost function is the cross-entropy error which the system defines between the estimated probabilities and the “true” sleep distribution. Given a dataset of N training samples the cross-entropy cost function is defined as follows:

$$J = - {\sum }_{i = 1}^{N} {t}_{i} log ( {y}_{i} ) + ( 1 - {t}_{i} ) log ( 1 - {y}_{i} )$$

where tᵢ is the true sleep probability for training sample i that could be either 0 or 1, yᵢ is the predicted probability which can take any value between 0 and 1. Minimizing the negative log likelihood cost function is equivalent to maximizing the likelihood of the correct probability.

During training, the cost function is minimized by tuning the model parameters so that the inputs corresponding to a sleep occurrence results in an output probability of close to 1 and inputs that are not associated with the occurrence of sleep results in an output probability of close to 0.

The built model, when fed by new inputs, outputs the probability of the sleep occurrence which could vary from 0 to 1. The model is updated over time based on the new user inputs and/or feedback data, and sensor data regarding sleep.

Other MLMs that can be used for this purpose can include naive Bayes, probabilistic decision tree and probabilistic support vector machines classifiers. Other structures of neural networks can also be incorporated such as recurrent neural networks, radial basis neural networks, etc.

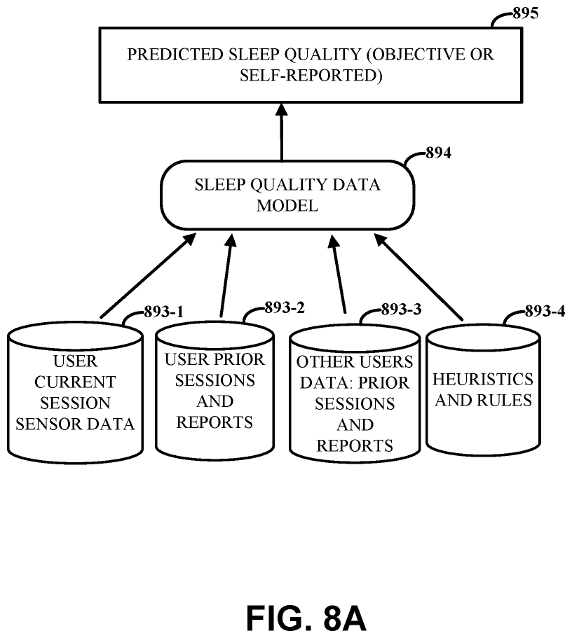

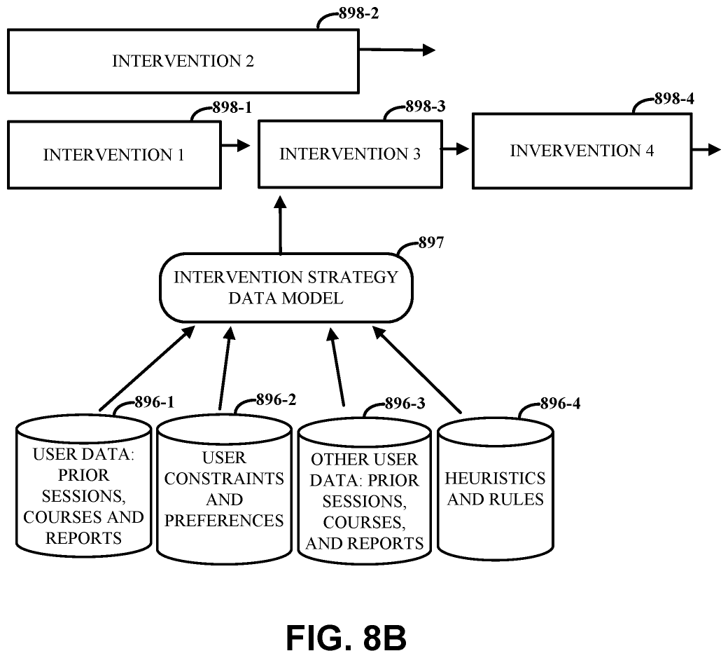

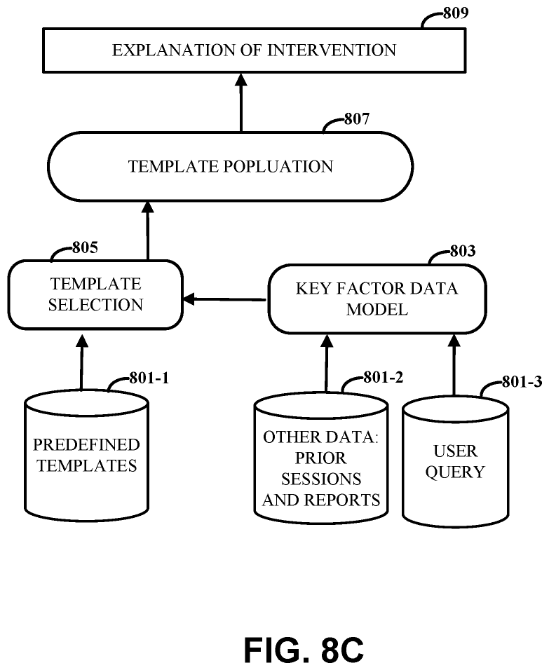

FIGS. 8A-8C illustrate different examples of predictive data models, in accordance with various embodiments. The different examples can include different implementations of the predictive data model as previously illustrated by FIG. 1.

As shown by FIG. 8A, different multivariable time series measurements from sensors (e.g., instruments) may be used to predict a sleep quality, which may be subjective and/or objective, for a current sleep session in progress. The input data 893-1, 893-2, 893-3, 893-4 to the sleep quality predictive data model 894 can include current user sensor data 893-1, prior user sensor data 893-2, other user data 893-3, and heuristics and rules 893-4. The current user sensor data 893-1 can include sensor measurements obtained from the user in the current sleep session which is on-going and can reflect a psychophysiological state of the user (e.g., autonomic functioning using HRV measures) and environmental circumstances (e.g., external or air temperature). The prior user sensor data 893-1 can include sensor measurements obtained from the user in prior sleep sessions, and/or objective and/or user self-reports of sleep quality from the prior sleep sessions. The other user data 893-3 can include sensor measurements obtained from a plurality of other users during prior sleep sessions, along with objective and/or user self-reports of sleep quality from the prior sleep sessions. The heuristics and rules 893-4 can include background information and knowledge, which can be codified as “if X then Y” rules.

The input data 893-1, 893-2, 893-3, 893-4 is provided to the sleep quality predictive data model 894 and the sleep quality predictive data model 894 provides an output 895. The output 895 may include a predicted value of a user self-report of sleep quality of the current sleep session, up to the current point and/or a predicted objective sleep quality value for the current sleep session, up to the current point. An objective sleep quality value can consist of an objectively derived value and/or several objectively derived values, such as a weighted combination. In some embodiments, the heuristics and rules 893-4 can provide a starting point and which is updated using the other input data 893-1, 893-2, 893-3 to obtain accurate and stable predictions as early in the current sleep session as possible. The output 895 can be used to determine if adjustments are to be made to the sleep intervention strategy to increase sleep quality.

As shown by FIG. 8B, different sleep intervention strategies can be used to reduce pre-sleep stress and/or improve sleep quality. The input data 896-1, 896-2, 896-3, 896-4 to the intervention strategy predictive data model 897 can include user data 896-1, user constraints and preferences 896-2, other user data 896-3, and heuristics and rules 893-6. The user data 896-1 can include sensor measurements obtained from the user in prior sleep sessions, prior sleep intervention strategies and/or objective and/or user self-reports of sleep quality from the prior sleep sessions. The user constraints and preferences 896 can include user constraints and preferences for intervention actions specific to the particular user. The other user data 896-3 can include sensor measurements obtained from a plurality of other users during prior sleep sessions, along with objective, prior sleep intervention strategies used for the users, and/or user self-reports of sleep quality from the prior sleep sessions. The heuristics and rules 896-4 can include background information and rules for intervention actions to maximize sleep quality.

The inputs data 896-1, 896-2, 896-3, 896-4 4 is provided to the intervention strategy predictive data model 897 and the intervention strategy predictive data model 897 provides an output, which in the example includes a plurality of intervention actions 898-1, 898-2, 898-3, 898-4 forming the sleep intervention strategy. The output may include recommended course of intervention actions to apply for an upcoming sleep session, which forms the sleep intervention strategy. The interventions may occur in parallel, simultaneously, and/or for variable durations.

As shown by FIG. 8C, users can select a specific intervention action in a given sleep intervention strategy, and can be provided with an explanation for why the intervention action was selected. The input data 801-1, 801-2, 801-3 can include predefined explanation templates 801-1, other data 801-2, and user query 801-3. The predefined explanation templates 801-1 can include templates consisting of language and charts suitable for use as a basis for the explanation. The other data 801-2 can include records of the user's session data (e.g., sensor data, strategies, and self-reports), records of other users' session data, and heurists and rules that encompass correlations between the interventions and self-reports. The user query 801-3 can include user selected subset of intervention actions of the sleep intervention strategy selected, which may be associated with prior or future sleep session.

The input data of other data 801-2 and the user query 801-3 is provided to the key factor predictive data model 803 which outputs key factors used to select the subset of intervention actions, which is input to the template selection data model 805. The template selection data model 805 pairs the input key factors with a template from the predefined fined explanation templates 801-1, and outputs the paired factors with the template to the template population data model 807 that populates the factors from the analysis into the template and outputs 809 a language based explanation (e.g., “selected X because it gave a boost in Y for similar users”) and can further output visualizations and/or plots.

The above described systems and computer-readable instructions can be used to track various factors of sleep for a user and to improve sleep for the user by generating a predictive data model which is dynamically updated over time. Based on the dynamic predictive model, the system is used to predict occurrence of a sleep transition, to increase the probability by developing and implementing a sleep invention strategy.

### More Detailed/Experimental Embodiments

Embodiments in accordance with the present disclosure include systems, devices, and methods involving management of sleep for one or a more users. Specific embodiments are directed to an acute psychophysiological state manipulation and sleep management system and to particular implementations of the same.

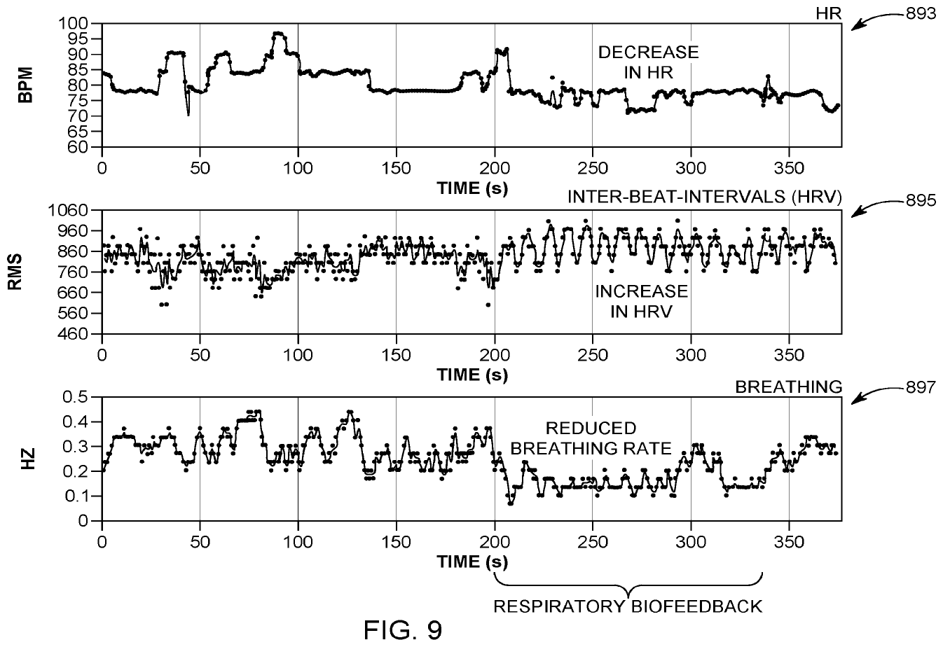

FIG. 9 shows a sample effect of the intervention on heartrate, HRV over time, and the use of an intervention planner to generate sleep intervention strategy for any particular user, in accordance with various embodiments.

More specifically, FIG. 9 shows the acute effect of virtual reality respiratory biofeedback on physiological arousal during a daytime relaxation test using data from a 49-year-old woman with sleep difficulties during a virtual reality relaxation test. The woman was sitting in a reclining position. After starting respiratory biofeedback (˜200 seconds after the beginning of the test), the subject was able to reduce her breathing rate at approximately 0.1 Hz (6 breath per minute), as shown by graph 897, leading to an improvement in HRV as noticeable from the inter-beat-interval, as shown by graph 895, and a reduction in HR, as shown by graph 893. The data shows how a behavioral intervention (in this case a virtual reality respiratory biofeedback) can acutely (within seconds) modulate the user's physiological state during while awake.

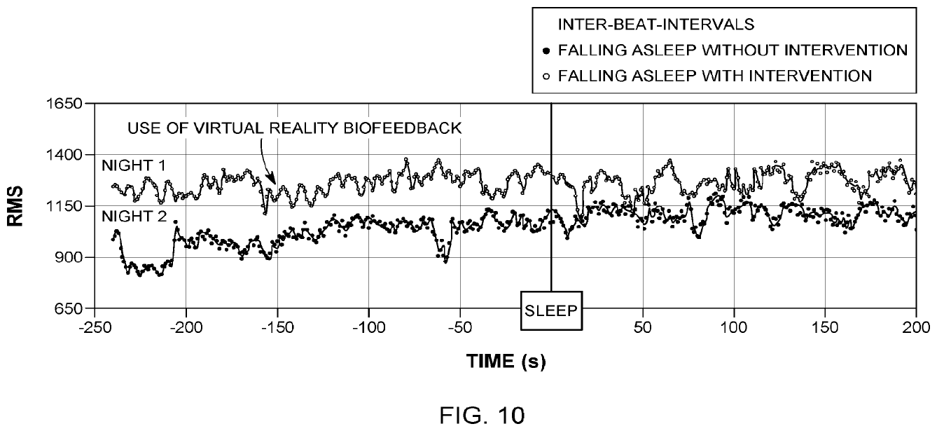

FIG. 10 shows inter-beat interval times for one falling asleep with and without intervention (in this case, virtual reality biofeedback), in accordance with various embodiments. For example, FIG. 10 illustrates the physiological deactivation (here reflected by enhanced inter-beat intervals, e.g., reduction in heart rate) while performing virtual reality respiratory biofeedback across the wake-to-sleep transition using data from a 53-year-old woman with severe insomnia. After an adaptation night, the subject spent two nights in the laboratory, on one night she received a virtual reality biofeedback intervention to facilitate her sleep. Compared to the control night, the use of virtual reality respiratory biofeedback resulted in a lengthening of the inter-beat-intervals (reduction in FIR) in the pre-sleep period, allowing the subject to approach sleep in a state of physiological relaxation. The data shows the feasibility of using a behavioral intervention (in this case a virtual reality respiratory biofeedback) across the whole wake-to-sleep transition to enhance relaxation and guide people to sleep.

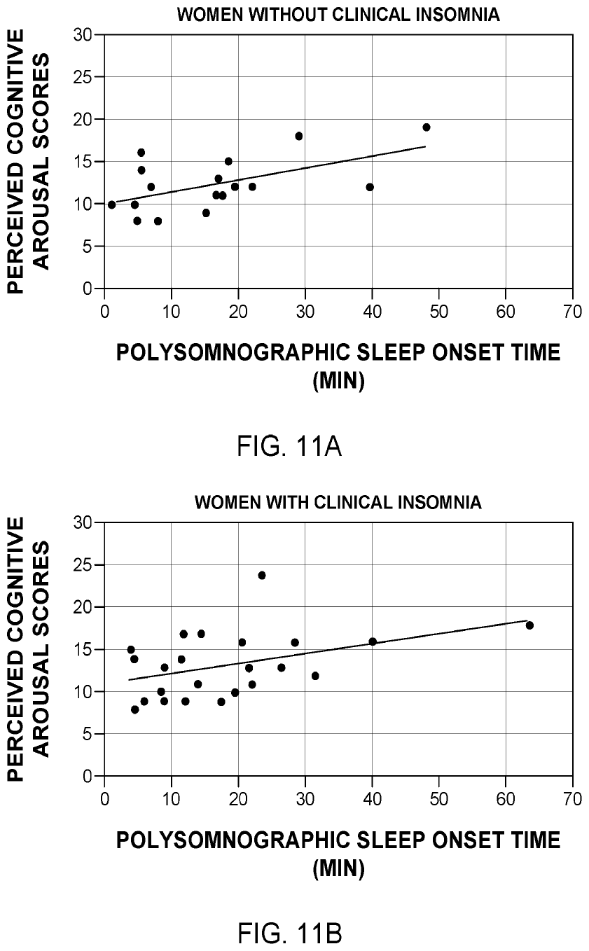

FIGS. 11A-11B show the group level relationship between perceived pre-sleep cognitive arousal and subsequent time spent falling asleep, for those with and without insomnia, in accordance with various embodiments. For example, FIGS. 11A-11B illustrate example pre-sleep self-report cognitive arousal is related to the objective time individuals spend falling asleep using data from twenty-five women, between the ages of 43 and 57, with clinical insomnia, as shown by FIG. 11B, and seventeen women without clinical insomnia, as shown by FIG. 11A. Bedtime cognitive arousal was measured using a standard questionnaire after a stress anticipation procedure. Specifically, the women were told that on the next day they would have to give a speech, simulating a job interview, in front of a committee that will evaluate their performance. The relation between stress induced cognitive arousal and objective time spent falling asleep is significantly evident (on a group level) in both women with and without insomnia (p<0.001). The data shows the importance of delivering focused interventions aiming to decrease pre-sleep arousal levels in order to improve sleep.

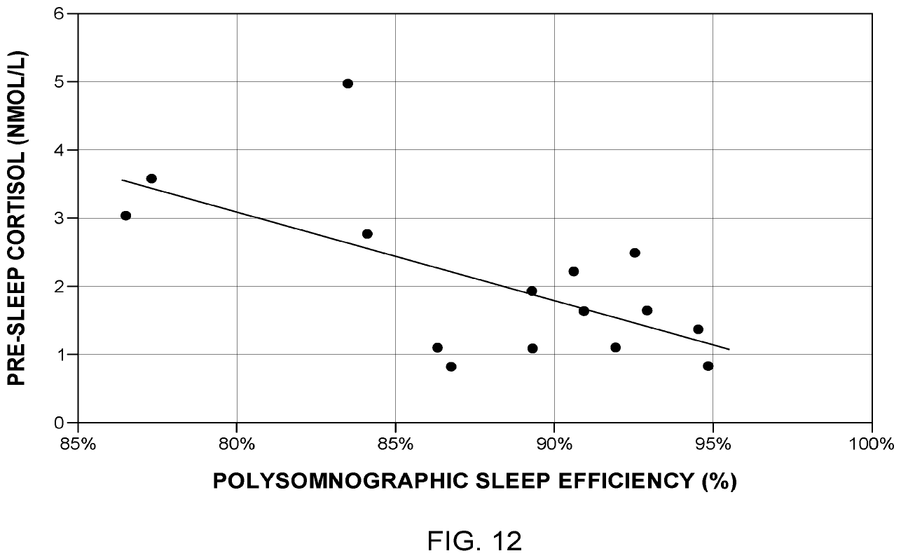

FIG. 12 shows a relation between physiological pre-sleep state of activation (cortisol levels) and night-time polysomnographic sleep efficiency, in accordance with various embodiments. For example, FIG. 12 shows an example relation between pre-sleep physiological stress level and polysomnographic sleep quality using data from eighteen healthy women between the ages of 45 and 51, without clinical sleep disturbances. The graph shows the relation between saliva cortisol collected immediately before bedtime and overnight polysomnographic sleep efficiency indicating that those women having greater bedtime cortisol level had lower sleep efficiency. The data suggest that pre-sleep stress levels affect nighttime sleep quality.

Different sources of information can be used to determine user levels of psychophysiological activation and, most importantly, to determine when this activation exceeds what is considered an “adaptive normal range”. This is an aspect of the disclosed system given that the recognition of the individual's arousal state at bedtime is useful to choosing the correct intervention and/or combination of interventions to decrease the arousal state to an optimal level.

The optimal level of pre-sleep activation can be determined based on the relation between pre-sleep physiological activation level and objective night-time sleep quality which is expected to be negative (more pre-sleep physiological activation, less sleep quality). Several other behavioral outcomes can be considered as well as user's perception of activation and other outcomes.

In this example, the objective sleep efficiency can be used to determine the optimal physiological level of pre-sleep activation the users need to achieve a certain sleep quality. This can be determined based on individuals' relationship between pre-sleep HRV state and subsequent sleep quality over time.

FIG. 13 shows a diagram of pre-sleep state effect on sleep, in accordance with various embodiments. In this case, a function was extracted that represent the relationship between pre-sleep HRV state and sleep quality that can be used to determine the threshold which discriminates the user's normal pre-sleep hyperarousal state from a lowered more optimal pre-sleep arousal state. The function can be determined on individual-based and/or on population-based criteria. Here a simple hypothetical relation is shown; this relation can change over time and can be influenced by multiple factors.

Resting HRV can be obtained by processing beat-to-beat variation in heart rate (low HRV is an index of poor autonomic functioning). It can be calculated over a period of time (e.g., five min) such as when the individual is lying down in bed before sleep.

Sleep efficiency was determined as the amount of time slept divided by the total time spent in bed and multiply by 100. Table 2 provides an example of 10 consecutive nights from a hypothetical user.

TABLE 2

| | Night | Night | Night | Night | Night | Night | Night | Night | Night | Night |
|---|---|---|---|---|---|---|---|---|---|---|
| User#1 | 1 | 2 | 3 | 4 | 5 | 6 | 7 | 8 | 9 | 10 |
| Pre-sleep | 176 | 200 | 59 | 300 | 200 | 180 | 89 | 330 | 150 | 45 |
| (Resting heart | | | | | | | | | | |
| rate variability) | | | | | | | | | | |
| Sleep efficiency | 78 | 89 | 55 | 99 | 88 | 79 | 65 | 100 | 70 | 50 |

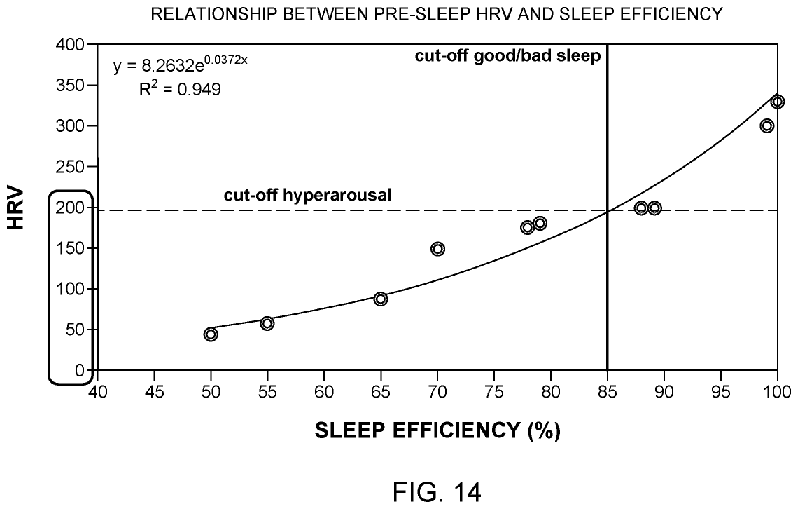

FIG. 14 illustrates a theoretical plot of the relationship between pre-sleep physiological autonomic activation (e.g., HRV), and subsequent night-time sleep efficiency, in accordance with various embodiments. For example, FIG. 14 shows an example of ten consecutive nights from a hypothetical user. In the example shown in FIG. 14, the user is considered in a hyperarousal state for HRV<200 based on the evidence that with HRV<200 the user will most likely have a bad night sleep (sleep efficiency<85%). In this case the intervention will aim to increase the HRV to values greater than 200. The curve (pre-sleep HRV and sleep efficiency) will be constantly updated day by day and resulting in potentially different thresholds.

Self-report tools (e.g., visual analog scale [VAS], questionnaires, etc.) can be also implemented. For example, to calculate fixed threshold for the adaptive normal range of users' perceived activation, the threshold can be set based on individuals' perception of altered psychophysiological state. For example, individuals can rate their level of anxiety on a 1-100 mm VAS (ranging from ‘extremely low’ to ‘extremely high’) and subsequently rate if this activation is considered “exceeding what they consider normal”. Based on population averaged responses, adaptive normal range thresholds can be determined and stratified based on individual demography.

Various embodiments are implemented in accordance with the underlying Provisional Application (Ser. No. 63/045,304), entitled “AI SLEEPZZZ,” filed Jun. 29, 2020, to which benefit is claimed and which are both fully incorporated herein by reference for their general and specific teachings. For instance, embodiments herein and/or in the provisional application can be combined in varying degrees (including wholly). Reference can also be made to the experimental teachings and underlying references provided in the underlying provisional application. Embodiments discussed in the Provisional Application are not intended, in any way, to be limiting to the overall technical disclosure, or to any part of the claimed disclosure unless specifically noted.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and can be abbreviated as“/”.

Although various illustrative embodiments are described above, any of a number of changes can be made to various embodiments without departing from the scope of the invention as described by the claims. For example, although described or shown with respect to one embodiment, the features and elements so described or shown can apply to other embodiments. As a further example, the order in which various described method steps are performed can often be changed in alternative embodiments, and in other alternative embodiments one or more method steps can be skipped altogether. Optional features of various device and system embodiments can be included in some embodiments and not in others. Therefore, the foregoing description is provided primarily for exemplary purposes and should not be interpreted to limit the scope of the invention as it is set forth in the claims.

The skilled artisan would recognize that various terminology as used in the Specification (including claims) connote a plain meaning in the art unless otherwise indicated. As examples, the Specification describes and/or illustrates aspects useful for implementing the claimed disclosure by way of various circuits or circuitry which can be illustrated as or using terms such as blocks, modules, device, system, unit, controller, and/or other circuit-type depictions. Such circuits or circuitry are used together with other elements to exemplify how certain embodiments can be carried out in the form or structures, steps, functions, operations, activities, etc. For example, in certain of the above-discussed embodiments, one or more modules are discrete logic circuits or programmable logic circuits configured and arranged for implementing these operations/activities, as may be carried out in the approaches shown in the figures. In certain embodiments, such a programmable circuit is one or more computer circuits, including memory circuitry for storing and accessing a program to be executed as a set (or sets) of instructions (and/or to be used as configuration data to define how the programmable circuit is to perform), and an algorithm or process as described herein used by the programmable circuit to perform the related steps, functions, operations, activities, etc. Depending on the application, the instructions (and/or configuration data) can be configured for implementation in logic circuitry, with the instructions (whether characterized in the form of object code, firmware or software) stored in and accessible from a memory (circuit).

Various embodiments described above, may be implemented together and/or in other manners. One or more of the items depicted in the present disclosure can also be implemented separately or in a more integrated manner, or removed and/or rendered as inoperable in certain cases, as is useful in accordance with particular applications. In view of the description herein, those skilled in the art will recognize that many changes may be made thereto without departing from the spirit and scope of the present disclosure.

What is Claimed is:

1. A system comprising:
memory circuitry to store a predictive data model indicative of different patterns and probabilities of a user transitioning from an awake state to a sleep state; and
processing circuitry to:
detect, using data indicative of a current psychophysiological state of the user, a pattern among the different patterns of the predictive data model that is indicative of a probability of the user transitioning from the awake state to the sleep state at a date and time;
based on the detected pattern, select an intervention action predicted to increase the probability of the user transitioning to the sleep state at the date and time; and
communicate a message indicative of the intervention action to the user.

2. The system of claim 1, wherein the processing circuitry is to detect the pattern by identifying, in the data, a feature set from among a plurality of feature sets and selecting a sub-model of the predictive data model using the feature set, the predictive data model including a plurality of sub-models that indicate the probability of the user transitioning to the sleep state in response to different intervention actions, and the plurality of sub-models being associated with a particular feature set of the plurality of feature sets.

3. The system of claim 2, wherein the plurality of sub-models are associated with different time frames, and each feature of the feature set has a weight associated with the probability of the user transitioning to the sleep state.

4. The system of claim 1, wherein the processing circuitry is to revise the predictive data model based on feedback data which is indicative of whether the user transitions to the sleep state responsive to the intervention action.

5. The system of claim 4, wherein the processing circuitry is to receive the feedback data in real time, and in response to the feedback data and the revised predictive data model, to communicate another message indicative of a revised intervention action.

6. The system of claim 4, wherein the processing circuitry is to receive the feedback data, and in response to the received feedback data:
identify features from the feedback data;
identify whether the user exhibits a response to the intervention action that is anticipated by the predictive data model to increase the probability based on the identified features; and
in response to an unexpected response, revise the predictive data model for the user and as associated with the detected pattern.

7. The system of claim 1, wherein the intervention action is part of a sleep intervention strategy that includes a plurality of intervention actions, the plurality of intervention actions being selected from a group consisting of: a behavioral intervention action, a cognitive intervention action, a neuromodulation action, an environmental change, a sensory action, and combinations thereof.

8. The system of claim 7, wherein the processing circuitry is to communicate the message indicative of the sleep intervention strategy and which includes an order of the plurality of intervention actions.

9. The system of claim 7, wherein the processing circuitry is to communicate a plurality of messages, including the message, that are indicative of the plurality of intervention actions, each of the plurality of messages being selected from a group consisting of: a message displayed to the user that instructs the user to take a respective intervention action, and a message to another device to automatically cause a respective intervention action to occur at a particular time in accordance with the sleep intervention strategy.

10. The system of claim 1, further including input circuitry to receive the data indicative of the current psychophysiological state of the user, the input circuitry including a wearable physiological sensor to sense a physiological signal from the user and another sensor to sense an atmospheric measurement.

11. The system of claim 1, further including input circuitry to receive the data indicative of the current psychophysiological state of the user, wherein the data received is selected from a group consisting of: schedule or calendar data, stress level, general mood, dietary data, health information, exercise data, sleep data, and a combination thereof.

12. A non-transitory computer-readable storage medium comprising instructions that when executed cause processing circuitry to:
identify a feature set among a plurality of feature sets from data, the data being indicative of a current psychophysiological state of a user;
based on the identified feature set, detect a pattern associated with a predictive data model that is indicative of a probability of the user transitioning from an awake state to a sleep state at a date and time;
based on the detected pattern and the predictive data model, communicate a message to the user indicative of an intervention action predicted to increase the probability of the user transitioning to the sleep state at the date and time; and
revise the predictive data model based on feedback data which is indicative of whether the user transitions to the sleep state in response to the intervention action.

13. The non-transitory computer-readable storage medium of claim 12, wherein the instructions to detect the pattern include instructions executable to select a sub-model of the predictive data model using the identified feature set, and the instructions being further executed to, in response to the feedback data and the revised predictive data model, communicate another message indicative of a revised intervention action.

14. The non-transitory computer-readable storage medium of claim 12, wherein the instructions to revise the predictive data model include instructions executable to revise a weight provided to the intervention action in response to the identified feature set for the user.

15. The non-transitory computer-readable storage medium of claim 12, wherein the instructions to revise the predictive data model include instructions executable to revise the predictive data model over time for the user based on the feedback data and additionally received feedback data that is indicative of different sleep intervention strategies and feature sets.

16. The non-transitory computer-readable storage medium of claim 12, wherein the intervention action is part of a sleep intervention strategy that includes a plurality of intervention actions, and the instructions are executable to:
communicate the message that is indicative of the sleep intervention strategy and including an order of the plurality of intervention actions; and
revise the predictive data model including revising one or more of the order of the plurality of intervention actions and the plurality of intervention actions.

17. A system comprising:
input circuitry to receive data indicative of a current psychophysiological state of a user;
memory circuitry to store a predictive data model indicative of different patterns and probabilities of the user transitioning from an awake state to a sleep state; and
processing circuitry to:
detect, using the data, a pattern among the different patterns of the predictive data model that is indicative of a probability of the user transitioning from the awake state to the sleep state at a date and time;
based on the detected pattern, identify a sleep intervention strategy including at least one intervention action predicted to increase the probability of the user transitioning to the sleep state at the date and time; and
communicate at least one message indicative of the at least one intervention action to the user.

18. The system of claim 17, wherein the memory circuitry includes instructions that when executed cause the processing circuitry to generate the predictive data model based on general population trends and publically available information and to revise the predictive data model for the user over time using feedback data indicative of success of different sleep intervention strategies for respective feature sets.

19. The system of claim 17, wherein the at least one message indicative of the at least one intervention action further includes an indication of an order and timing of the at least one intervention action.

20. The system of claim 17, wherein the processing circuitry is to communicate the at least one message to another device to automatically cause the at least one intervention action to occur at a particular time in accordance with the sleep intervention strategy.
